(19) United States
(12) Patent Application Publication
(10) Pub. No.: US 2005/0095222 A1
(43) Pub. Date: May 5, 2005

(54) Allergen inhibitor, allergen-inhibiting method, allergen-inhibiting fiber and allergen-inhibiting sheet

(76) Inventors: Taro Suzuki, Osaka (JP); Mitsuhito Teramoto, Osaka (JP); Yoji Fujimori, Hachioji-city (JP)

Correspondence Address: KUBOVCIK & KUBOVCIK, SUITE 710, 900 17TH STREET NW, WASHINGTON, DC, 20006, US

(21) Appl. No.: 10/695,459

(22) Filed: Oct. 29, 2003

(51) Int. Cl. A61K 31/765; A61K 31/185

(52) U.S. Cl. 424/78.37; 514/553

(57) ABSTRACT

The allergen inhibitor of this invention comprises at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof.

### FIELD OF THE INVENTION

The present invention relates to an allergen inhibitor which inhibits allergens such as pollens from plants such as cedar etc. and allergens from mites and room dust from reacting with specific antibodies, without coloring or polluting daily articles, a method of inhibiting allergens, allergen-inhibiting fibers and an allergen-inhibiting sheet having an allergen-inhibiting effect.

### BACKGROUND OF THE INVENTION

In recent years, allergic diseases such as atopic dermatitis, bronchial asthma and allergic rhinitis come to be problematic. These allergic diseases are caused mainly by allergens increasing a living space such as allergens from mites existing abundantly in houses, particularly dermatophagoide allergens (Der 1, Der 2) existing abundantly in room dust and cedar pollen allergens (Crij1, Crij2) floating abundantly in the air in spring.

The allergic disease caused by dermatophagoide allergens cannot be fundamentally solved by exterminating dermatophagoides because dead bodies of dermatophagoides act as allergens. As cedar pollen allergen, Crij1 is a glycoprotein having a molecular weight of about 40 kDa, and Crij2 is a glycoprotein having a molecular weight of about 37 kDa. The cedar pollen allergen upon adhering to a nasal mucous membrane is recognized as a foreign body to induce an inflammation reaction.

For reducing symptoms of the allergic disease or preventing new allergic symptoms, it is therefore necessary to completely eliminate allergens from a living space or to inactivate allergens by inactivation.

Allergen is a protein. It is therefore estimated that when allergen is denatured by heating or with a strong acid or alkali, the allergen is not allergenic. However, the allergen is so highly stable that it cannot be easily denatured by an oxidizing agent, a reducing agent, heating, an alkali or an acid usable safely in home (The Journal of Immunology, Vol. 144:1353-1360).

Denaturation of allergens can, depending on conditions, cause damage to places polluted with allergens, that is, daily articles such as tatami mats, carpets, floors, furniture (sofas, cloth-backed chairs, tables), bedding (beds, futon, sheet), articles in a car (sheet, child sheet), kitchen articles, articles for babies, curtains, wallpapers, towels, clothing, stuffed toys, other fiber products and an air cleaner (main body and a filter).

Accordingly, a method of chemically denaturing the surface of an allergen molecule under relatively mild conditions has been devised. For example, there have been proposed a method of inhibiting allergens with tannic acid used in tanning a raw hide or the like (JP-A 61-44821), a method of inhibiting allergens with a tea extract or the like (JP-A 6-279273), a method of inhibiting allergens with a hydroxybenzoic acid-type compound or a salt thereof (JP-A 11-292714) etc., and the allergen-inhibiting effects of these compounds have also been confirmed.

However, almost all these compounds are one kind of polyphenol and are thus colored, to cause the problem of coloring the daily articles described above.

### SUMMARY OF THE INVENTION

This invention provides an allergen inhibitor which can inhibit allergens effectively without polluting or damaging the surface of daily articles to which allergen adhere, a method of inhibiting allergens, allergen-inhibiting fibers and an allergen-inhibiting sheet having an allergen-inhibiting effect.

The allergen inhibitor of this invention comprises at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof.

The allergen inhibitor of this invention comprises a phosphate and either or both of zinc sulfate and lead acetate.

The allergen inhibitor of this invention further comprises an aqueous solution containing aluminum sulfate and at least one sulfate selected from the group consisting of sodium sulfate, potassium sulfate, ammonium sulfate and thallium sulfate.

The method of inhibiting allergens according to this invention comprises supplying at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof, in an object where allergens exist to inhibit the allergens.

The method of inhibiting allergens according to this invention comprises supplying an aqueous solution containing aluminum sulfate and at least one sulfate selected from the group consisting of sodium sulfate, potassium sulfate, ammonium sulfate and thallium sulfate, in an object where allergens exist to inhibit the allergens.

The allergen-inhibiting fibers of this invention comprise an allergen inhibitor contained in fibers.

The allergen-inhibiting sheet of this invention comprises an allergen inhibitor comprising at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof contained in a base sheet.

The allergen-inhibiting sheet of this invention comprises an allergen inhibitor comprising a phosphate and either or both of zinc sulfate and lead acetate contained in a base sheet.

The allergen-inhibiting sheet of this invention comprises an allergen inhibitor comprising an aqueous solution containing aluminum sulfate and at least one sulfate selected from the group consisting of sodium sulfate, potassium sulfate, ammonium sulfate and thallium sulfate contained in a base sheet.

### ADVANTAGES OF INVENTION

The allergen inhibitor of this invention exhibits an excellent allergen-inhibiting effect without polluting or damaging daily articles.

The allergen-inhibiting method of this invention comprises supplying a compound having an allergen-inhibiting effect (allergen inhibitor) in an object where allergens exist thereby inhibiting the allergens effectively without polluting or damaging the object.

The allergen-inhibiting fibers of this invention exhibit an excellent allergen-inhibiting effect without necessitating treatment for inhibiting allergens. Accordingly, the allergen-inhibiting fibers can be used to produce a fiber product having an excellent allergen-inhibiting effect.

Finally, the allergen-inhibiting sheet of this invention can eliminate allergens and simultaneously exhibit an allergen-inhibiting effect by merely wiping a region where allergens are to be inhibited with the allergen-inhibiting sheet thereby exhibiting the allergen-inhibiting effect more effectively.

### DETAILED DESCRIPTION OF THE PREFERRED EMBODIMENTS

The allergen inhibitor of this invention comprises at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof.

First, the aromatic hydroxy compound is described. The aromatic hydroxy compound is not particularly limited insofar as it has an aromatic hydroxyl group and exhibits an allergen-inhibiting effect, and examples thereof include an aromatic hydroxy compound obtained by polymerizing or copolymerizing a monomer containing at least one of substituent groups represented by the following general formulas (1) to (6), such as an aromatic hydroxy compound having, in a linear polymer, at least one of substituent groups represented by the general formulas (1) to (6); an aromatic heterocyclic hydroxy compound; and an aromatic hydroxyl compound obtained by polymerizing or copolymerizing a monomer having an aromatic heterocyclic hydroxy group, such as an aromatic hydroxy compound having, in a linear polymer, an aromatic heterocyclic hydroxy group as a substituent group, among which the aromatic hydroxy compound having, in a linear polymer, at least one of substituent groups represented by the general formulas (1) to (6) below and the aromatic hydroxy compound having, in a linear polymer, an aromatic heterocyclic hydroxy group as a substituent group are preferable.

In the present invention, the allergen-inhibiting effect refers to inhibition of the reactivity of allergens to specific antibodies by denaturing or adsorbing allergens such as dermatophagoide allergens (Der 1, Der 2), cedar pollen allergens floating in the air (Crij1, Crij2) and allergens attributable to cats and dogs (Can f 1, Fel d 1).

First, the aromatic hydroxy compound obtained by polymerizing or copolymerizing a monomer containing at least one of substituent groups represented by the general formulas (1) to (6) is described. The substituent group(s) in this aromatic hydroxy compound is represented by the following general formulas (1) to (6):

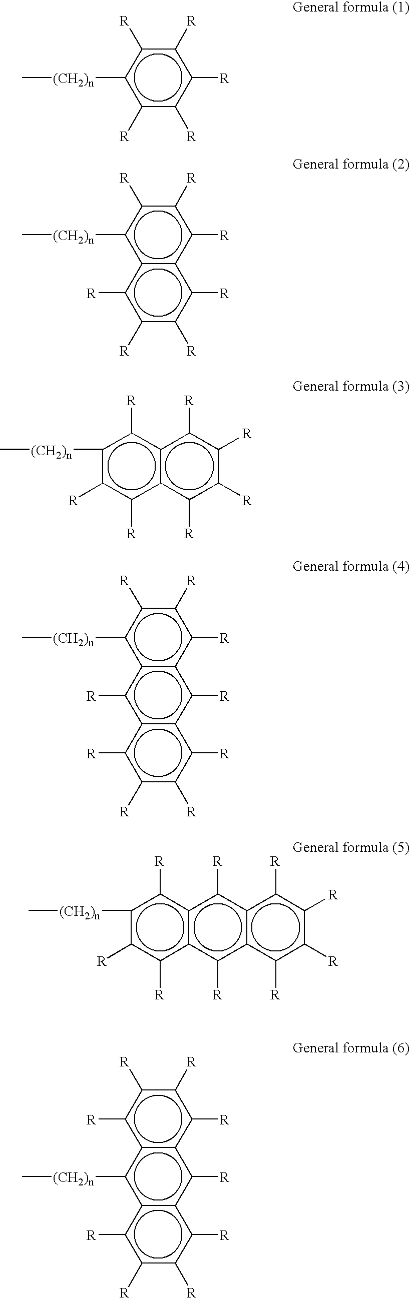

In the general formulas (1) to (6), n is an integer of 0 to 5. This is because when n is 6 or more, the allergen-inhibiting effect exhibited by the substituent groups represented by the general formulas (1) to (6) is insufficient.

Each of substituent groups R is a hydrogen atom or a hydroxyl group. At least one of the substituent groups R should be a hydroxyl group in order to permit the aromatic hydroxy compound to exhibit an allergen inhibiting effect. However, the number of hydroxyl groups is preferably 1 because when the number of hydroxyl groups is too high, a material to which the allergen inhibitor was applied can be easily colored or discolored. That is, it is preferable that only one of the substituent groups R is a hydroxyl group, while all the other substituent groups R are hydrogen atoms.

Preferably a hydroxyl group is bound thereto at a position of minimum steric hindrance; for example, a hydroxyl group is bound thereto preferably at the para-position in the general formula (1).

The monomer containing at least one of substituent groups represented by the general formulas (1) to (6) is not particularly limited insofar as it has substituent group(s) represented by the general formulas (1) to (6), and examples thereof include monomers having a monovalent phenol group, such as vinyl phenol, tyrosine, 1,2-di(4-hydroxyphenyl)ethene (general formula (7)).

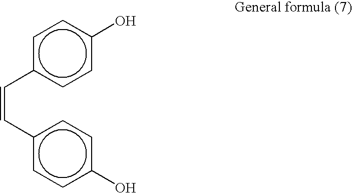

The monomer containing at least one of substituent groups represented by the general formulas (1) to (6), preferably a monomer having one or more monovalent phenol groups, may be copolymerized with a monomer copolymerizable therewith insofar as the allergen-inhibiting effect of the aromatic hydroxy compound is not inhibited.

The monomer copolymerizable therewith includes, for example, ethylene, acrylate, methacrylate, methyl acrylate, methyl methacrylate, hydroxyethyl acrylate, hydroxyethyl methacrylate, hydroxypropyl acrylate, hydroxypropyl methacrylate, styrene etc.

The linear polymer to which the substituent groups represented by the general formulas (1) to (6) are bound includes, but is not limited to, vinyl polymers, polyesters, polyamides etc. The chemical bonds between the linear polymer and the substituent groups represented by the formulas (1) to (6) include, but are not limited to, a carbon-carbon bond, an ester linkage, an ether linkage and an amide linkage etc.

The aromatic hydroxy compound having, in the linear polymer, at least one of substituent groups represented by the general formulas (1) to (6) includes, for example, (1) a polymer or copolymer of monomers each containing at least one of substituent groups represented by the general formulas (1) to (6), (2) a copolymer of a monomer containing at least one of substituent groups represented by the general formulas (1) to (6) and a monomer copolymerizable therewith and the like.

Preferable examples of the aromatic hydroxy compound having, in the linear polymer, at least one of substituent groups represented by the general formulas (1) to (6) include poly(vinyl 3,4,5-hydroxybenzoate), polyvinyl phenol, polytyrosine, poly(1-vinyl-5-hydroxynaphthalene), poly(1-vinyl-6-hydroxynaphthalene) and poly(1-vinyl-5-hydroxyanthracene).

The molecular weight of the aromatic hydroxy compound obtained by polymerizing the monomers described above is not particularly limited, but is preferably an aromatic hydroxy compound obtained by polymerizing two or more monomers, more preferably an aromatic hydroxy compound obtained by polymerizing five or more monomers.

The aromatic heterocyclic hydroxy compound is not particularly limited insofar as it exerts an inhibitive effect on allergens, and examples thereof include 2-hydroxy furan, 2-hydroxy thiophene, hydroxy benzofuran and 3-hydroxy pyridine etc.

Now, the aromatic hydroxy compound obtained by polymerizing or copolymerizing a monomer having an aromatic heterocyclic hydroxy group, such as an aromatic hydroxy compound having, in a linear polymer, an aromatic heterocyclic hydroxy group as a substituent group is described.

The aromatic heterocyclic hydroxy group includes a group comprising a hydroxy group bound to a heterocyclic skeleton such as thiophene or furan (general formulas (8) and (9)), a group comprising a hydroxy group bound to a skeleton containing a heterocycle and an aromatic ring (general formula (10)), a group comprising a hydroxy group and an alkyl group having 5 or less carbon atoms bound to a heterocyclic skeleton, a group comprising a hydroxy group and an alkyl group having 5 or less carbon atoms bound to a skeleton containing a heterocycle and an aromatic ring etc.;

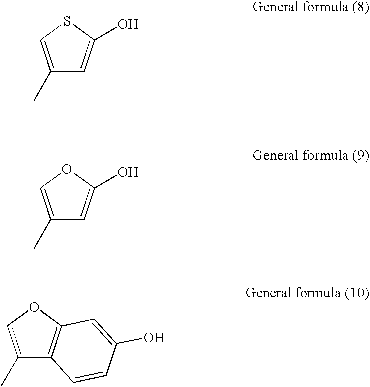

The linear polymer having an aromatic heterocyclic hydroxy group bound thereto includes, but is not limited to, vinyl polymers, polyesters polyamides etc. The chemical bond between the linear polymer and the aromatic heterocyclic hydroxy group includes, but is not limited to, a carbon-carbon bond, an ester linkage, an ether linkage an amide linkage etc.

The compound obtained by polymerizing or copolymerizing a monomer having an aromatic heterocyclic hydroxy group includes, for example, (1) a polymer or copolymer of monomers each having an aromatic heterocyclic hydroxy group and (2) a copolymer of a monomer having an aromatic heterocyclic hydroxy group and a monomer copolymerizable therewith and the like.

The monomer copolymerizable with a monomer having an aromatic heterocyclic hydroxy group includes, for example, ethylene, acrylate, methacrylate, methyl acrylate, methyl methacrylate, hydroxyethyl acrylate, hydroxyethyl methacrylate, hydroxypropyl acrylate, hydroxypropyl methacrylate styrene etc.

The amount of the aromatic hydroxy compound used is suitably determined depending on condition of the allergen inhibitor used and is not particularly limited. Specifically, when a carpet where mites live is sprayed therewith, the amount of the allergen inhibitor may be regulated such that 1 m² carpet is sprayed with 1 mg to 10 g of the aromatic hydroxy compound.

This is because when the amount of the aromatic hydroxy compound is low, the allergen-inhibiting effect is not exhibited, while when the amount of the aromatic hydroxy compound is high, the aromatic hydroxy compound may be precipitated to contaminate an object of allergen described later.

The alkali metal carbonate includes, for example, alkali metal carbonates such as lithium, sodium, potassium, rubidium, cesium and francium carbonates, preferably sodium carbonate, sodium bicarbonate and potassium carbonate.

The amount of the alkali metal carbonate used is suitably determined depending on condition of the allergen inhibitor used and is not particularly limited. Specifically, when a carpet where mites live is sprayed therewith, the amount of the allergen inhibitor may be regulated such that 1 m² carpet is sprayed with 1 mg to 10 g of the alkali metal carbonate.

This is because when the amount of the alkali metal carbonate is low, the allergen-inhibiting effect is not exhibited, while when the amount of the alkali metal carbonate is high, the alkali metal carbonate may be precipitated to contaminate an object of allergen.

The above-mentioned alum includes double salts consisting of monovalent ion (alkali metal, thallium, ammonium etc.) sulfate and aluminum sulfate. The alum may also be a double salt wherein aluminum sulfate is replaced by chromium sulfate, iron sulfate or the like.

The alum is preferably aluminum potassium sulfate or sodium aluminum sulfate. Aluminum potassium sulfate having a particularly high allergen-inhibiting effect is used mainly in the form of a hydrate with 12H₂O(AlK(SO₄)₂.12H₂O) or an anhydride (AlK(SO₄)₂), but may be in the form of a partial hydrate occurring in a process where the hydrate gradually releases water molecules.

Some alums such as potassium alums are highly safe substances which are also designated as food additives and cosmetic materials. When the alum is used in a carpet to inhibit allergens, the carpet does not feel sticky nor is foamed, and can thus feel good in use without foaming. In respect of safety and feel in use, the alum is used preferably for the purpose of inhibiting allergens in carpets.

The amount of alum used is suitably regulated depending on condition of the allergen inhibitor used and is not particularly limited. Specifically, when a carpet where mites live is sprayed therewith, the amount of the allergen inhibitor is regulated such that 1 m² carpet is sprayed with 1 mg to 10 g alum.

This is because when the amount of alum used is low, the allergen-inhibiting effect is not exhibited, while when the amount of alum is high, the alum may be precipitated to contaminate daily articles.

The lauryl benzene sulfonate, lauryl sulfate and polyoxyethylene lauryl ether sulfate include lithium, sodium, potassium and magnesium metal salts, ammonium salts, amine salts such as triethanol amine salts etc., among which the sodium salt and triethanol amine salt are preferable.

The amount of the lauryl benzene sulfonate, lauryl sulfate or polyoxyethylene lauryl ether sulfate used is suitably determined depending on condition of the allergen inhibitor used and is not particularly limited. Specifically, when a carpet where mites live is sprayed therewith, the amount of the allergen inhibitor may be regulated such that 1 m² carpet is sprayed with 1 mg to 10 g lauryl benzene sulfonate, lauryl sulfate or polyoxyethylene lauryl ether sulfate.

This is because when the amount of the lauryl benzene sulfonate, lauryl sulfate or polyoxyethylene lauryl ether sulfate used is low, the allergen-inhibiting effect is not exhibited, while when the amount of the lauryl benzene sulfonate, lauryl sulfate or polyoxyethylene lauryl ether sulfate used is high, the lauryl benzene sulfonate, lauryl sulfate or polyoxyethylene lauryl ether sulfate may be precipitated to contaminate daily articles.

The divalent or more sulfate having either or both of a polyoxyethylene chain and a polyethylene chain in the molecule thereof include, for example, lithium, sodium, potassium and magnesium metal salts, ammonium salts, and amine salts such as triethanol amine salt, among which the sodium salt and triethanol amine salt are preferable.

Specifically, the divalent or more sulfate having either or both of a polyoxyethylene chain and a polyethylene chain in the molecule thereof include a divalent or more benzene sulfonate having either or both of a polyoxyethylene chain and a polyethylene chain in the molecule thereof and tri(sodium lauryl sulfate)glycerin, among which the divalent or more benzene sulfonate having either or both of a polyoxyethylene chain and a polyethylene chain in the molecule thereof is preferable.

The divalent or more benzene sulfonate having either or both of a polyoxyethylene chain and a polyethylene chain in the molecule thereof includes, for example, alkyl diphenyl ether disulfonates such as disodium lauryl diphenyl ether disulfonate, alkyl triphenyl ether trisulfonates such as trisodium lauryl triphenyl ether sulfonate, polyoxyethylene diphenyl ether disulfonates such as disodium hexaoxyethylene diphenyl ether sulfonate etc., among which the alkyl diphenyl ether disulfonate is preferable.

The amount of the divalent or more sulfate having either or both of a polyoxyethylene chain and a polyethylene chain in the molecule thereof is suitably determined depending on condition of the allergen inhibitor used and is not particularly limited. Specifically, when a floor contaminated with allergens is sprayed therewith, the amount of the allergen inhibitor may be regulated such that 1 m² floor is sprayed with 1 mg to 10 g divalent or more sulfate having either or both of a polyoxyethylene chain and a polyethylene chain in the molecule thereof.

This is because when the amount of the divalent or more sulfate having either or both of a polyoxyethylene chain and a polyethylene chain in the molecule thereof is low, the allergen-inhibiting effect is not exhibited, while when the amount of the divalent or more sulfate having either or both of a polyoxyethylene chain and a polyethylene chain in the molecule thereof is high, the floor feels sticky and should be cleaned after use.

The allergen inhibitor may comprise a phosphate and either or both of zinc sulfate and lead acetate. That is, the allergen inhibitor may comprise (1) zinc sulfate and a phosphate, (2) lead acetate and a phosphate, and (3) zinc sulfate, lead acetate and a phosphate.

The phosphate refers to a salt which upon being dissolved in an aqueous solvent, forms a PO₄³⁻ ion, and examples thereof include sodium dihydrogen phosphate (monosodium phosphate), disodium hydrogen phosphate (disodium phosphate), potassium dihydrogen phosphate etc.

The above-mentioned zinc sulfate is used mainly in the form of a hydrate (with 7H₂O) or an anhydride, but may be in the form of a partial hydrate occurring in a process where the hydrate gradually releases water molecules.

Zinc sulfate is known as Chinese white, zinc white etc. and also described in the Japanese Pharmacopoeia. It is also a food additive which is highly safe and added to a food as a substitute for mother's milk in order to supply Zn as a trace metal element essential for human growth and health.

The above-mentioned lead acetate is used in the form of a hydrate (with 3H₂O) or an anhydride, but may be in the form of a partial hydrate occurring in a process where the hydrate gradually releases water molecules. Lead acetate has been known as sugar of lead for a long time and is also described in the Japanese Pharmacopoeia.

The amount of the phosphate used is preferably about 0.001 M or more in the form of a phosphate buffer. This is because when the amount of the phosphate is low, the allergen inhibitor may fail to exhibit an allergen-inhibiting effect. However, when the content of the phosphate is too high, an object of allergen may be contaminated with the phosphate, and thus the amount of the phosphate is preferably 0.001 to 0.1 M.

The amount of zinc sulfate used is suitably determined depending on condition of the allergen inhibitor used and is not particularly limited. Specifically, when a carpet where mites live is sprayed therewith, the amount of the allergen inhibitor may be regulated such that 1 m² carpet is sprayed with 0.1 mg or more in terms of the weight of zinc sulfate.7H₂O.

This is because when the amount of zinc sulfate is low, the allergen-inhibiting effect is not exhibited. However, when the amount of zinc sulfate is too high, an object of allergen may be contaminated with zinc sulfate, and thus the amount of zinc sulfate is preferably 0.1 mg to 1 g.

The amount of lead acetate used is suitably determined depending on condition of the allergen inhibitor used and is not particularly limited. Specifically, when a carpet where mites live is sprayed therewith, the amount of the allergen inhibitor may be regulated such that 1 m² carpet is sprayed with 0.1 mg to 1 g in terms of the weight of lead acetate.3H₂O.

This is because when the amount of lead acetate is low, the allergen-inhibiting effect is not exhibited, while when the amount of lead acetate is too high, an object of allergen may be contaminated with lead acetate.

The allergen inhibitor may be an aqueous solution containing aluminum sulfate and at least one sulfate selected from the group consisting of sodium sulfate, potassium sulfate, ammonium sulfate and thallium sulfate. This allergen inhibitor is excellent in stability without recrystallization of the solute in the aqueous solution at low temperatures.

The above-mentioned sodium sulfate is used mainly in the form of an anhydride (Na₂SO₄) or a hydrate with 10H₂O(Na₂SO₄.10H₂O), but may be in the form of a partial hydrate occurring in a process where the hydrate gradually releases water molecules. Sodium sulfate is a highly safe substance which is also used as a food additive and a starting material of cosmetics.

The above-mentioned potassium sulfate is used in the form of an anhydride (K₂SO₄) and described in the Japanese Pharmacopoeia, and used mainly in pharmaceutical preparations or fertilizers. Ammonium sulfate is used in the form of an anhydride [(NH₄)₂SO₄] and used as a food additive in a yeast food for bread, etc.

The above-mentioned aluminum sulfate is used mainly in the form of an anhydride [Al₂(SO₄)₃] or a hydrate with 18H₂O [Al₂(SO₄)₃.18H₂O], but may be in the form of a partial hydrate occurring in a process where the hydrate gradually releases water molecules. Aluminum sulfate is used widely as sulfuric acid band for tap water.

When sodium sulfate and aluminum sulfate are simultaneously used, the total concentration of sodium sulfate and aluminum sulfate is preferably 0.5 to 50% by weight.

This is because when the total concentration of sodium sulfate and aluminum sulfate is low, the allergen-inhibiting effect may not be exhibited, while when the total concentration of sodium sulfate and aluminum sulfate is high, the solute in an aqueous solution may be recrystallized in winter to deteriorate stability.

The various compounds referred to as the allergen inhibitor may be used alone or as a mixture of two or more thereof.

Now, use of the allergen inhibitor is described. Preferably, the allergen inhibitor of this invention excluding the allergen inhibitor comprising an aqueous solution containing aluminum sulfate and at least one sulfate selected from the group consisting of sodium sulfate, potassium sulfate, ammonium sulfate and thallium sulfate is dissolved or dispersed in a solvent to form an allergen inhibitor solution. The content of the allergen inhibitor in the allergen inhibitor solution is preferably 0.01 to 50% by weight.

The solvent includes, for example, water (preferably ion-exchanged water), alcohols (methyl alcohol, ethyl alcohol, propyl alcohol etc.), hydrocarbons (toluene, xylene, methyl naphthalene, kerosene, cyclohexane etc.), ethers (diethyl ether, tetrahydrofuran, dioxane etc.), ketones (acetone, methyl ethyl ketone etc.) and amides (N,N-dimethylformamide etc.).

The allergen inhibitor comprising an aqueous solution containing aluminum sulfate and at least one sulfate selected from the group consisting of sodium sulfate, potassium sulfate, ammonium sulfate and thallium sulfate is an aqueous solution. Accordingly, it is not necessary that this allergen inhibitor, unlike the above-mentioned allergen inhibitor, is dissolved in another solvent. The aqueous solution may be mixed with an alcohol. The alcohol includes methyl alcohol, ethyl alcohol, propyl alcohol etc.

The “allergen inhibitor solution” referred to hereinafter encompasses the “allergen inhibitor comprising an aqueous solution containing aluminum sulfate and at least one sulfate selected from the group consisting of sodium sulfate, potassium sulfate, ammonium sulfate and thallium sulfate”.

The allergen inhibitor may contain pharmaceutical additives such as a dispersant, an emulsifying agent, a wetting agent, a thickener, an antioxidant and an ultraviolet absorbing agent as well as a tickicide, an antimicrobial agent, an antifungal agent and a deodorant in such a range that the allergen-inhibiting effect of the allergen inhibitor is not inhibited.

The allergen inhibitor can be used by various methods of spraying, aerosol spraying, smoking or heating dissipation. By incorporating a water solvent, a oil solution, an emulsifier, a suspending agent or the like into the allergen inhibitor solution, the allergen inhibitor can be used by spraying. Spraying is a method of pressurizing the allergen inhibitor solution at general pressures to spray the allergen inhibitor.

By adding a solid carrier (talc, bentonite, clay, kaolin, diatomaceous earth, silica, vermiculite, pearlite etc.) to the spray-type allergen inhibitor, the allergen inhibitor can be used as an aerosol-type allergen inhibitor.

The aerosol-type allergen inhibitor is used by introducing the allergen inhibitor solution together with a propellant into a container to keep the solution in a compressed state, and spraying the allergen inhibitor by the pressure of the propellant. The propellant includes, for example, nitrogen, carbon dioxide gas, dimethyl ether, LPG etc.

The smoking-type allergen inhibitor can be produced by adding an oxygen generator (potassium perchlorate, potassium nitrate, potassium chlorate etc.), a combustible material (saccharides, starch etc.), a heating regulator (guanidine nitrate, nitroguanidine, guanyl urea phosphate etc.) and an oxygen generator-decomposing assistant (potassium chloride, copper oxide, chromium oxide, iron oxide, active carbon etc.) to the spray-type allergen inhibitor. The smoking-type allergen inhibitor is used by dispersing smoke formed by finely dividing the allergen inhibitor.

Instead of the allergen inhibitor used by spraying or dispersing it as described above, an allergen-inhibiting sheet may be formed by impregnating a base sheet with the allergen inhibitor. Preferably, the base sheet is impregnated with the allergen inhibitor in the form of an allergen inhibitor solution. The base sheet is not particularly limited insofar as it can be impregnated with the allergen inhibitor (allergen inhibitor solution), and the base sheet is a fiber aggregate or a foam, preferably a fiber aggregate.

This is because the fiber aggregate can be easily impregnated with the allergen inhibitor (allergen inhibitor solution), to maintain the allergen inhibitor and can smoothly supply the allergen inhibitor (allergen inhibitor solution) to an object where allergens are to be inhibited.

The fiber aggregate is not particularly limited, and examples thereof include woven or nonwoven fabric, knitting etc., preferably woven or nonwoven fabric, more preferably nonwoven fabric. The nonwoven fabric includes, but is not limited to, needle punched nonwoven fabric, spun bonded nonwoven fabric and water-jet confounded nonwoven fabric.

The fibers constituting the fiber aggregate include, for example, thermoplastic resin fibers such as polyester-based fibers, polyamide-based fibers and polyolefin fibers, composite fibers thereof, semi-synthetic fibers such as acetate, regenerated fibers such as cuprammonium and rayon, natural fiber such as cotton and cellulose, and mixed fibers of two or more thereof.

The fiber aggregate may be subjected to surface treatment with a surfactant, a oil solution or the like, or endowed with an electrostatically adsorbing effect by depolarization treatment to improve a function of eliminating allergens.

The foam includes foams of synthetic resins such as polyethylene, polypropylene and polystyrene, foams of naturally occurring polymers such as natural rubber, and molded products obtained by melting synthetic resin or a naturally occurring polymer by heat or with a solvent and then molding it so as to have pores, and the like.

The amount of the allergen inhibitor impregnated in the base sheet is preferably 0.1 to 100% by weight, more preferably 0.2 to 60% by weight, still more preferably 0.5 to 30% by weight based on the base material.

This is because when the amount of the allergen inhibitor is low, the allergen-inhibiting effect is not exhibited, while when the amount of the allergen inhibitor is high, the surface of an object of allergen upon wiping with the allergen-inhibiting sheet feels sticky or the allergen inhibitor may be precipitated to contaminate the object.

The amount of the allergen inhibitor solution impregnated in a base sheet is preferably 50 to 500% by weight, more preferably 100 to 400% by weight, based on the base material. This is because when the amount of the allergen inhibitor solution is low, the allergen inhibitor solution may not be sufficiently supplied into an object where allergens exist, thus failing to effectively exhibit its allergen-inhibiting effect, while when the amount of the allergen inhibitor solution is high, the surface of an object of allergen upon wiping with the allergen-inhibiting sheet can change its shape.

When the allergen-inhibiting sheet is used on a tatami mat, an oily substance-solubilizing solvent is preferably added to the allergen inhibitor in order to increase the allergen-inhibiting effect.

This is because allergens often adhere to the surface of a tatami mat not only by themselves but also together with an oil generated from the human body, and thus the oily substance is removed by dissolving it in an oily substance-solubilizing solvent, whereby the allergen inhibitor can act more effectively on allergens.

The oily substance-solubilizing solvent includes nonionic surfactants such as polyoxyethylene alkylene phenyl ether, anionic surfactants, cationic surfactants, amphoteric surfactants, glycerin and propylene glycol, and in consideration of foaming and detergency upon application to tatami mats, nonionic surfactants and propylene glycol are preferable. The amount of the oily substance-solubilizing solvent used can be suitably determined, but is preferably 0.1 to 200% by weight based on the base sheet.

The allergen-inhibiting sheet when stored or not used is placed preferably in an evaporation-preventing container or an evaporation-preventing bag in order to prevent evaporation of the allergen inhibitor.

The evaporation-preventing bag is not particularly limited, but is preferably a bag made of a composite film with high gas barrier properties gluing an aluminum foil stuck on a synthetic resin film toghther.

The divalent or more sulfate having either or both of a polyoxyethylene chain and a polyethylene chain in the molecule thereof is particularly poor in foaming and is thus preferably used in cleaner wax for floor or in a detergent for floor or tatami mat.

The cleaner wax for floor is produced for example by mixing wax, natural resin or synthetic resin with a solvent, a thickener, a surfactant, an emulsifier, water and if necessary a coloring matter, a masking agent, a plasticizer and an antistatic agent. The wax may be derived from plants, minerals or animals. The synthetic resin includes, for example, polyethylene, polyvinyl acetate, polyvinyl chloride, polystyrene, acrylic resin, polyalkylene glycol, polyphenylene ether, polyalkylene glycol etc.

The solvent includes, for example, mineral sprit, mineral terpene, solvent naphtha, terpene oil etc. The surfactant includes polyoxyethylene fatty esters etc. The emulsifier includes triethanolamine soap etc.

For the purpose of using it in a detergent for floor or tatami mat, the detergent for floor and tatami mat may be prepared by adding paraffin, naphthene, aromatic hydrocarbons, synthetic hydrocarbons, metallic soap, molybdenum disulfide, a surfactant, an antimicrobial agent and an antifungal agent to the allergen inhibitor.

Depending on the manner of using the allergen inhibitor, the inhibitor can be supplied by spraying, scattering or applying, in an object where allergens exist, that is, an object where allergens are to be inhibited (referred to hereinafter as “object of allergen”) to achieve inhibition of allergens.

The “inhibition of allergens” refers to inhibition of the reactivity of allergens to specific antibodies by denaturing or adsorbing allergens such as dermatophagoide allergens (Der 1, Der 2), cedar pollen allergens floating in the air (Crij1, Crij2) and allergens attributable to cats and dogs (Can f 1, Fel d 1).

Particularly, when the surface of an object of allergen is wiped with the allergen-inhibiting sheet, allergens on the surface of the object of allergen can be removed, and simultaneously, the reactivity, to specific antibodies, of allergens remaining on the surface of the object of allergen can be inhibited by the allergen inhibitor, whereby the allergen-inhibiting effect can be exhibited more effectively.

The object of allergen includes daily articles serving as a hotbed of allergen in a living space. The daily articles include, for example, tatami mats, carpets, floors, furniture (sofas, cloth-backed chairs, tables), bedding (beds, futon, sheet), articles in a car (sheet, child sheet), kitchen articles, articles for babies, curtains, wallpapers, towels, clothing, stuffed toys, fiber products and an air cleaner (main body and a filter). The allergen inhibitor can also exhibit an allergen-inhibiting effect by adding to a detergent, a softening agent, etc.

The allergen as the object of the allergen inhibitor of this invention includes animal allergens and plant allergens such as pollens. Animal allergens against which the allergen inhibitor is particularly effective can be any types of acarian allergens (acarians, creatures of arthropod-arachnida-acarina, which are divided into roughly 7 suborders, that is, notostigmata represented by Opilioacaridae, tetrastigmata represented by Holothyridae, metastigmata represented by Ixodes ovatus and Argas japonicus, mesostigmata represented by Ornithonyssus bacoti and Dermanyssus hirundinis, prostigmata represented by Cheyletus malaccensis and Tarsonemus granarius, astigmata represented by dermatophagoides such as Dermatophagoides farinae, and Tyrophagus putrescentiae, and cryptostigmata represented by Haplochthonius simplex and Cosmochthonius reticulatus), but the allergen inhibitor is particularly effective against dermatophagoides causing allergic diseases, which exist abundantly in room dust, particularly in bedding.

Depending on the object of allergen, the allergen inhibitor described above can be supplied to inhibit the reactivity, to specific antibodies, of allergens existing in the object of allergen.

Fibers may be endowed with an allergen-inhibiting effect by incorporation of the allergen inhibitor described later, including the above-described allergen inhibitor, into the fibers as allergen-inhibiting fibers. The allergen-inhibiting fibers can be used to produce daily articles endowed with an allergen-inhibiting effect.

The allergen-inhibiting fibers are described in more detail. The allergen-inhibiting fibers comprise the allergen inhibitor incorporated into fibers, and the allergen-inhibiting fibers are preferably those capable of exhibiting an allergen-inhibiting effect in an atmosphere in an absolute humidity of not higher than 50 g/m³.

The allergen inhibitor contained in the fibers includes not only the above-described allergen inhibitor but also plant extracts such as tannic acid and catechin, and is preferably the above-described allergen inhibitor.

The above-described inhibitor is specifically an allergen inhibitor comprising at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof; an allergen inhibitor comprising a phosphate and either or both of zinc sulfate and lead acetate; and an allergen inhibitor comprising an aqueous solution containing aluminum sulfate and at least one sulfate selected from the group consisting of sodium sulfate, potassium sulfate, ammonium sulfate and thallium sulfate.

The fibers are not particularly limited insofar as they can contain the allergen inhibitor, and examples thereof include synthetic fibers such as polyester fibers, nylon fibers, acryl-based fibers and polyolefin-based fibers, semi-synthetic fibers such as acetate fibers, regenerated fibers such as cuprammonium rayon and rayon, natural fiber such as cotton, hemp, wool and silk, composite fibers thereof, and mixture thereof.

The fibers may be those capable of forming a reaction field capable of causing interaction with allergen by gathering water molecules in the air. The reaction field capable of causing interaction with allergen by gathering water molecules in the air is a reaction field where a certain chemical interaction is exerted to inhibit the antigenicity of an antigenic moiety (epitope) of allergen, which refers for example to a reaction field where an electrochemical transition state such as an ionized state is stabilized to lower the barrier energy of the transition state for chemical reaction thereby advancing a natural chemical reaction. Usually, water in a liquid state is necessary for reducing the energy barrier of a transition state to initiate a chemical reaction. The fibers capable of forming a reaction field capable of causing interaction with allergen by gathering water molecules in the air can form such a site by gathering water molecules in the air, thus making a procedure such as splashing of water unnecessary.

The fibers capable of forming a reaction field capable of causing interaction with allergen by gathering water molecules in the air are preferably fibers containing a hygroscopic compound, and hygroscopic fibers.

The hygroscopic compound includes, for example, polyether such as polyethylene glycol, polypropylene glycol and polyoxymethylene; polyalcohol such as polyvinyl alcohol; polymer salts such as poly(sodium acrylate); polymer acids such as polyacrylic acid, among which the polyether is preferable because it is excellent in hygroscopicity and easily releases its entrapped water molecules into a system.

The content of the hygroscopic compound in the allergen-inhibiting fibers is preferably 0.01 to 300% by weight, more preferably 0.1 to 30% by weight, still more preferably 0.1 to 10% by weight.

This is because when the content of the hygroscopic compound is low, the allergen-inhibiting effect may not be exhibited, while when the content of the hygroscopic compound is high, the allergen-inhibiting effect is adversely deteriorated.

The hygroscopic fibers include, for example, natural fibers such as wool, silk, hemp and cotton, regenerated fibers such as cuprammonium rayon and rayon, semi-synthetic fibers such as acetate fibers and synthetic fibers such as nylon fibers, among which cotton is preferable. The hygroscopic fibers may be mixed with other fibers.

The fibers may be those having improved moisture absorption and desorption, prepared from generally used semi-synthetic or synthetic fibers by subjecting them to any one of the following processes. The process includes (1) process of changing the surface shape or sectional shape of fibers, (2) process of changing the sectional shape of fibers, (3) process of making fibers porous, (3) process which involves copolymerizing a moisture absorbable/desorbing polymer, (4) process which involves incorporating a moisture absorbable/desorbing polymer into fibers, (5) process of making a core/sheath structure, and (5) process for bonding a moisture absorbable/desorbing compound to the surface of fibers.

The surface of fibers capable of forming a reaction field capable of causing interaction with allergen by gathering water molecules in the air is regulated preferably in the range of pH 6 or more in order to further improve the allergen-inhibiting effect of the allergen-inhibiting fibers.

The pH of the surface of the fibers is determined by dropping pure water, preferably ion-exchanged water, onto the surface of the fibers, then leaving the fibers for about 15 minutes to moisten their surface and measuring the pH by a pH test paper.

The surface of fibers capable of forming a reaction field capable of causing interaction with allergen by gathering water molecules in the air preferably incorporates an alkali metal oxide or alkaline earth metal oxide, an alkali metal hydroxide or alkaline earth metal hydroxide.

The alkali metal includes lithium, sodium, potassium, rubidium, cesium and francium, and the alkaline earth metal includes beryllium, magnesium, calcium, strontium, barium and radium.

The content of the alkali metal oxide or alkaline earth metal oxide and the alkali metal hydroxide or alkaline earth metal hydroxide in the fibers is preferably 0.001 to 30% by weight, more preferably 0.01 to 3% by weight, still more preferably 0.1 to 1% by weight.

This is because when the content of the oxide or hydroxide is low, the allergen-inhibiting fibers may fail to exhibit an effect of improving the allergen-inhibiting effect, while when the content of the oxide or hydroxide is high, the fibers may be damaged.

Now, the method of incorporating the allergen inhibitor into fibers is described. The method of incorporating the allergen inhibitor into fibers includes a method of chemically bonding the allergen inhibitor to fibers and a method of physically fixing or mixing the allergen inhibitor in fibers.

First, the method of chemically bonding the allergen inhibitor to fibers includes a method of chemically bonding the allergen inhibitor to fibers by graft reaction.

The method of graft reaction includes, but is not limited to, the following methods: (1) a method of graft polymerization by creating a polymerization initiation site on a fiber-forming backbone polymer to form the allergen inhibitor as a branch polymer by means of polymerization, and (2) a coupling method (polymer reaction) of chemically bonding the allergen inhibitor to fibers by polymer reaction.

Specifically, the graft polymerization method includes: (1) a method involving polymerization by radicals formed in chain of transfer reaction to fibers, (2) a method of polymerization on fibers by free radicals formed by allowing a reducing compound such as an alcohol, thiol or amine to act on secondary cerium salt or silver sulfate to form an oxidation-reduction system (redox system), (3) a method which involves irradiating fibers with γ-rays or accelerated electron beam in the coexistence of the fibers and a monomer serving as a starting material of the allergen inhibitor, (4) a method of polymerization by irradiating only fibers with γ-rays or accelerated electron beam and then adding a monomer serving as a starting material of the allergen inhibitor, (5) a method which involves oxidizing a fiber-forming polymer and introducing a peroxy group or converting an amino group on a side chain of the polymer into a diazo group, to start polymerization from the introduced group as a polymerization initiation site, and (6) a method of utilizing the polymerization initiation reaction of epoxy, lactam, polar vinyl monomer etc. by a side-chain active group such as a hydroxyl group, amino group, carboxyl group etc.

The graft polymerization method is specifically as follows: a) a method of graft polymerization by milling cellulose in a monomer serving as a starting material of the allergen inhibitor to form free radicals, b) a method of graft polymerization by using a monomer as a starting material of the allergen inhibitor and a cellulose derivative (e.g., mercaptoethyl cellulose) which as fiber, has a group easily undergoing chain of transfer, c) a method of graft polymerization by oxidizing ozone or peroxides to form radicals, d) a method of graft polymerization by introducing a double bond of allyl ether, vinyl ether or methacrylate into a side chain of cellulose, e) a method of graft polymerization by irradiating fibers with ultraviolet rays in the presence of a photosensitizer such as sodium anthraquinone-2,7-disulfonate, and f) a method of electrochemical graft polymerization by winding fibers on a cathode, adding a monomer as a starting material of the allergen inhibitor to dilute sulfuric acid, and applying external voltage.

In consideration of graft polymerization onto fibers, the following methods are preferable: g) a method of graft polymerization by heating fibers applied with glycidyl methacrylate (GMA) and benzoyl peroxide, in a solution of a monomer serving as a starting material of the allergen inhibitor and h) a method of graft polymerization by adding a monomer as a starting material of the allergen inhibitor to an aqueous dispersion of benzoyl peroxide, a surfactant (nonionic or anionic surfactant) and monochlorobenzene and dipping fibers such as polyester-based fibers therein, followed by heating.

The coupling method can be carried out by general methods including, for example, (1) chain of transfer reaction onto C—H, oxidation reaction and substitution reaction, (2) addition reaction and oxidation reaction of a double bond, (3) esterification, etherification, conversion into acetal of a hydroxyl group; substitution reaction, addition reaction, hydrolysis reaction of an ester or amide group; substitution reaction, and elimination reaction of a halogen group, and (4) substitution reaction (halogenation, nitration, sulfonation, chloromethylation) of an aromatic ring.

The monomer used in graft reaction may be a reactive or polymerizable monomer which is preferably a monomer having at least one of substituent groups represented by the general formulas (1) to (6), or may be an oligomer of this monomer. In the general formulas (1) to (6), R is a hydrogen or a hydroxyl group, at least one R is a hydroxyl group, and n is 0 to 5.

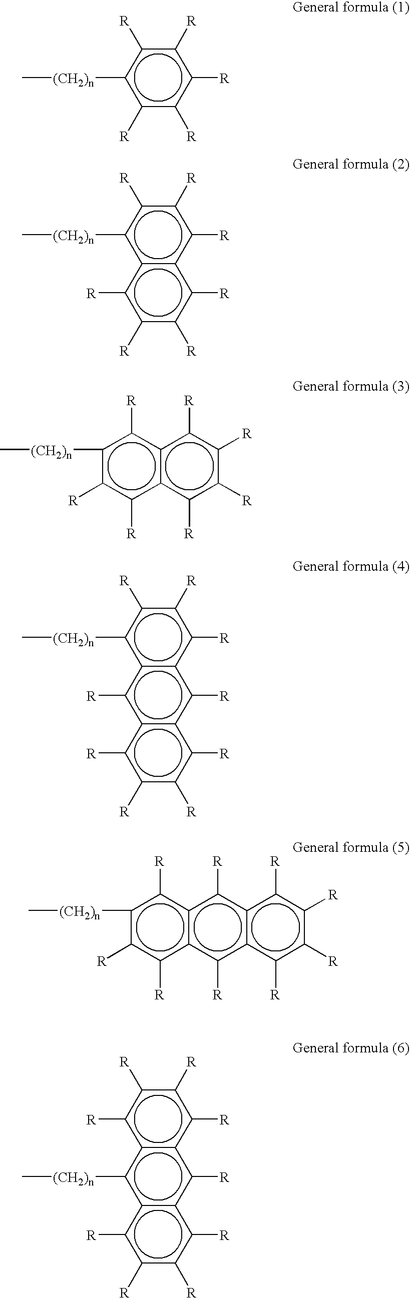

The monomer used in graft reaction may be a reactive or polymerizable monomer, and when a monomer having at least one of substituent groups represented by the general formulas (1) to (6) or an oligomer of the monomer is used, a hydrophilic monomer is preferably copolymerized therewith in polymerization. By copolymerizing the hydrophilic monomer, the allergen inhibitor can smoothly act on allergens thereby effectively exhibiting an allergen-inhibiting effect. The hydrophilic monomer includes, but is not limited to, vinyl acetate and 2-hydroxyethyl methacrylate (HEMA).

Now, the method of physically fixing the allergen inhibitor in fibers is described in detail. The method of physically fixing the allergen inhibitor in fibers includes, for example, (1) a method of impregnating fibers with the allergen inhibitor solution by dipping the fibers in the allergen inhibitor solution, (2) a method of applying the allergen inhibitor solution onto the surface of fibers, (3) a method of fixing the allergen inhibitor via a binder to fibers by dipping the fibers in a binder containing the allergen inhibitor dissolved or dispersed therein, and (4) a method of fixing the allergen inhibitor via a binder to fibers by applying a binder containing the allergen inhibitor dissolved or dispersed therein onto the surface of the fibers. In the methods (1) and (2), the following binder may be contained in the allergen inhibitor solution.

The binder is not particularly limited insofar as the allergen inhibitor can be fixed via the binder to the surface of fibers, and examples of synthetic resin binders include one-pack urethane resin, two-pack urethane resin, acrylic resin, urethane acrylate resin, polyester resin, unsaturated polyester resin, alkyd resin, vinyl acetate resin, vinyl chloride resin, epoxy resin and epoxy acrylate resin.

By adding a hydrophilic material to the allergen inhibitor solution or the binder, the allergen inhibitor can smoothly act on allergens thereby effectively exhibiting an allergen-inhibiting effect. The hydrophilic material includes, for example, cellulose, polyvinyl alcohol etc.

The method of mixing the allergen inhibitor in fibers includes a melt spinning method, a wet spinning method, a dry spinning method, an emulsion spinning method, a conjugate spinning method and a stretching method, as well as a method of highly stretching a bar-shaped polymer containing the allergen inhibitor and a method which involves interfacial polymerization.

The melt spinning method is a method of producing the allergen-inhibiting fibers by heat-melting a starting material of fibers, kneading the starting material of fibers in a melted state with the allergen inhibitor having a decomposition temperature higher than the melting point of the starting material of fibers, extruding the mixture from a spinneret having a desired pore to an inert cooling medium (for example the air, nitrogen, water etc.) and solidified by cooling therein.

The wet spinning method is a method of producing the allergen-inhibiting fibers by dissolving a starting material of fibers in a solvent to form a solution, then dissolving the allergen inhibitor or dispersing and mixing it in the solution to prepare a starting spinning dope, and extruding the starting spinning dope through a spinneret.

The dry spinning method is a method of producing the allergen-inhibiting fibers by dissolving a starting material of fibers in a volatile solvent to form a solution, then dissolving the allergen inhibitor or dispersing and mixing it in the solution to prepare a starting spinning dope, and extruding the starting spinning dope through a spinneret.

The emulsion spinning method is a method of producing the allergen-inhibiting fibers by preparing an emulsion, suspension or slurry of a starting material of fibers, then dissolving the allergen inhibitor or dispersing and mixing it in the solution to prepare a starting spinning dope, and spinning the starting spinning dope in the same manner as in the wet or dry spinning method.

The conjugate spinning method is a method of producing the allergen-inhibiting fibers by dissolving the allergen inhibitor or dispersing and mixing it in separately melted two or more starting fiber materials to prepare melts containing the allergen inhibitor in a melted state, mixing the melts just before a spinneret and spinning the mixture through the spinneret.

The stretching method is a method of producing the allergen-inhibiting fibers by stretching a film containing the allergen inhibitor, cutting the film into strips, stretching the strips and thermally fixing them.

The allergen-inhibiting fibers produced in the manner described above can be used to produce daily articles, for example, carpets, furniture (sofas, cloth-backed chairs, tables), bedding (beds, futon, sheet), articles in a car (sheet, child sheet), kitchen articles, articles for babies, curtains, wallpapers, towels, clothing, stuffed toys, fiber products, and a filter in an air cleaner.

The daily articles produced from the allergen-inhibiting fibers can exhibit an allergen-inhibiting effect to inhibit the reactivity of allergens to specific antibodies without separately supplying the allergen inhibitor.

While the allergen-inhibiting fibers are used, the allergen-inhibiting effect is gradually decreased. The allergen-inhibiting effect of the allergen-inhibiting fibers can be recovered by various methods.

The method of recovering the allergen-inhibiting effect of the allergen-inhibiting fibers includes a method of bleeding the allergen inhibitor out of the fibers and a method of eliminating inactivated allergens accumulated on the surface of the fibers.

Specifically, there is a method of washing the allergen-inhibiting fibers with a liquid, a method of heating the allergen-inhibiting fibers, or a method of cleaning the surface of the allergen-inhibiting fibers by suction with a cleaner.

The liquid used in washing the allergen-inhibiting fibers is not particularly limited insofar as the allergen-inhibiting fibers are not damaged, and examples thereof includes water, alcohols (methyl alcohol, ethyl alcohol, propyl alcohol etc.), hydrocarbons (toluene, xylene, methyl naphthalene, kerosene, cyclohexane etc.), ethers (diethyl ether, tetrahydrofuran, dioxane etc.), ketones (acetone, methyl ethyl ketone etc.) and amides (N,N-dimethylformamide etc.), among which water and an alcohol are preferable because of easy handling. To increase the washing effect, a generally used surfactant may also be concomitantly used.

The heating temperature of the fibers is not particularly limited insofar as the allergen-inhibiting fibers are not damaged, and the fibers can be heated by any methods including, for example, a method of directly heating the allergen-inhibiting fibers, a method of heating the allergen-inhibiting fibers dipped in a solvent, and a method of heating the allergen-inhibiting fibers by sunlight.

### EMBODIMENTS

### Examples 1 to 7

Poly-4-vinyl phenol (weight-average molecular weight (Mw) 8000, manufactured by Aldrich), poly-4-vinyl phenol (poly-p-vinyl phenol) (Mw 20000, manufactured by Aldrich), or poly-L-tyrosine (Mw 15000 to 36000, manufactured by ICN Biomedicals) was used as the allergen inhibitor, and ethyl alcohol and ion-exchanged water were used as the solvent to prepare allergen inhibitor solutions. The resulting allergen inhibitor solution was introduced into a trigger-type spray container (about 0.8 ml spray by spraying once). The amounts of the allergen inhibitor and the solvent used are shown in Table 1.

### Examples 8 to 19

Sodium carbonate (Wako Pure Chemical Industries, Ltd.), potassium carbonate, sodium bicarbonate, alum (aluminum potassium sulfate (food additive) manufactured by Wako Pure Chemical Industries, Ltd.), sodium lauryl sulfate (Wako Pure Chemical Industries, Ltd.), lauryl sulfate triethanol amine (Kao Corporation), sodium lauryl benzene sulfonate (Wako Pure Chemical Industries, Ltd.) or sodium polyoxyethylene lauryl ether sulfate (Kao Corporation) was used as the allergen inhibitor, and ion-exchanged water was used as the solvent to prepare allergen inhibitor solutions. The resulting allergen inhibitor solution was introduced into a trigger-type spray container (about 0.8 ml spray by spraying once). The amounts of the allergen inhibitor and the solvent used are shown in Table 1. In Example 12, however, the amount of sodium carbonate used was 1.5% by weight, and the amount of sodium bicarbonate was 1.5% by weight.

### Examples 20 to 28

Zinc sulfate.7H₂O (Wako Pure Chemical Industries, Ltd.), lead(II)acetate.2H₂O (Wako Pure Chemical Industries, Ltd.), a phosphate buffer (pH 7.35) at a concentration of 0.01 M prepared by dissolving phosphates i.e. 0.01 M monosodium phosphate.2H₂O (Kanto Kagaku) and 0.01 M disodium phosphate.12H₂O (Wako Pure Chemical Industries, Ltd.) in ion-exchanged water and a phosphate buffer (pH 7.35) at a concentration of 0.001 M prepared by dissolving phosphates i.e. 0.001 M monosodium phosphate.2H₂O (Kanto Kagaku) and 0.001 M disodium phosphate.12H₂O (Wako Pure Chemical Industries, Ltd.) in ion-exchanged water were used to produce allergen inhibitor solutions. The resulting allergen inhibitor solution was introduced into a trigger-type spray container (about 0.8 ml spray by spraying once). The amounts of the starting materials used are shown in Table 1.

### Comparative Examples 1 to 3

Ion-exchanged water, ethyl alcohol and a phosphate buffer were mixed or used alone in the formulations shown in Table 1 to produce allergen inhibitor solutions. The resulting allergen inhibitor solution was introduced into a trigger-type spray container (about 0.8 ml spray by spraying once). The amounts of the starting materials used are shown in Table 1.

### Comparative Examples 4 and 5

Tannic acid (Tokyo Kasei Kogyo Co., Ltd.) and succinic acid (Tokyo Kasei Kogyo Co., Ltd.) were used as the allergen inhibitor, and ethyl alcohol and ion-exchanged water were used as the solvent to prepare allergen inhibitor solutions. The resulting allergen inhibitor solution was introduced into a trigger-type spray container (about 0.8 ml spray by spraying once). The amounts of the allergen inhibitor and the solvent used are shown in Table 1.

### Comparative Example 6

An aqueous sodium hydroxide solution, pH 12 (solution prepared by adding 0.1 N hydrochloric acid to 0.1 N sodium hydroxide and adjusting the solution to pH 12 by titration) was used as the allergen inhibitor solution. The allergen inhibitor solution was introduced into a trigger-type spray container (about 0.8 ml spray by spraying once).

### Comparative Example 7

An allergen inhibitor solution was produced in the same manner as in Example 20 except that ion-exchanged water was used in place of the phosphate buffer. The resulting allergen inhibitor solution was introduced into a trigger-type spray container (about 0.8 ml spray by spraying once). The amounts of the starting materials used are shown in Table 1.

### Comparative Example 8

An allergen inhibitor solution was produced in the same manner as in Example 24 except that ion-exchanged water was used in place of the phosphate buffer. The resulting allergen inhibitor solution was introduced into a trigger-type spray container (about 0.8 ml spray by spraying once). The amounts of the starting materials used are shown in Table 1.

The allergen inhibitive action and coloration of the allergen inhibitors were measured by methods described later, and the results are shown in Table 1. The pH values of the allergen inhibitor solutions are shown in Table 1.

Allergen Inhibitive Action

A new carpet was sprayed in an amount of 50 μg/m² with acarian antigen-containing dust (acarian antigen was quantified by Mighty Checker™ manufactured by Shinto Fine Co., Ltd.), and then the bottom of the carpet was vibrated with a vibrator to penetrate the dust into the carpet, whereby a polluted carpet was prepared.

The upper surface of the polluted carpet was sprayed 4 times with the allergen inhibitor solution from the spray container. After the carpet was left for 2 hours, a dust-collecting filter (Mighty Felt, Dust Filter, Dust Sampler™ manufactured by Shinto Fine Co., Ltd.) was attached to a cleaner (Maihime 510 SC-9™ manufactured by Sanyo Electric Co., Ltd.). Using the cleaner, the dust was collected by suction for 1 minute from an arbitrary 1 m² area in the upper surface of the polluted carpet. Allergen components were extracted from the collected dust by an allergen extractor (Mighty Checker™ manufactured by Shinto Fine Co., Ltd.), and the amount of the allergens was determined. The results are shown in Table 1.

Judgment Criteria
++: Acarian allergen level>35 μg/m²
+: Acarian allergen level 10 μg/m²
±: Acarian allergen level 5 μg/m²
−: Acarian allergen level<1 μg/m²

Coloration

A new white carpet was uniformly sprayed 4 times with the allergen inhibitor solution, and whether the carpet was colored or not was visually observed. ◯ was given to no coloration, and x was given to coloration.

TABLE 1

| | | | Ion-exchanged | Ethyl | Phosphate | | | Allergen | |
|---|---|---|---|---|---|---|---|---|---|
| | Allergen inhibitor | | water | alcohol | buffer | | | inhibitive | |
| | Compound | Weight % | Weight % | Weight % | 0.01M | 0.001M | pH | action | Coloration |
| Example 1 | Poly-4-vinyl phenol | 3 | 48.5 | 48.5 | — | — | 6.1 | — | ∘ |
| | (Mw: 8000) | | | | | | | | |
| Example 2 | Poly-4-vinyl phenol | 1 | 49.5 | 49.5 | — | — | 6.9 | ± | ∘ |
| | (Mw: 8000) | | | | | | | | |
| Example 3 | poly-4-vinyl phenol | 3 | 48.5 | 48.5 | — | — | 7.1 | — | ∘ |
| | (Mw: 20000) | | | | | | | | |
| Example 4 | Poly-4-vinyl phenol | 1 | 49.5 | 49.5 | — | — | 7.3 | — | ∘ |
| | (Mw: 20000) | | | | | | | | |
| Example 5 | Poly-4-vinyl phenol | 0.3 | 49.85 | 49.85 | — | — | 7.4 | ± | ∘ |
| | (Mw: 20000) | | | | | | | | |
| Example 6 | Poly-L-tyrosine | 1 | 49.5 | 49.5 | — | — | 7.2 | — | ∘ |
| Example 7 | Poly-L-tyrosine | 0.3 | 49.85 | 49.85 | — | — | 7.4 | ± | ∘ |
| Example 8 | Sodium carbonate | 3 | 97 | — | — | — | 10.9 | — | ∘ |
| Example 9 | Sodium carbonate | 0.3 | 99.7 | — | — | — | 10.4 | ± | ∘ |
| Example 10 | Potassium carbonate | 3 | 97 | — | — | — | 11.0 | — | ∘ |
| Example 11 | Potassium carbonate | 0.3 | 99.7 | — | — | — | 10.5 | ± | ∘ |
| Example 12 | Sodium carbonate/ | 3 | 97 | — | — | — | 9.4 | ± | ∘ |
| | sodium bicarbonate | | | | | | | | |
| Example 13 | Alum | 3 | 97 | — | — | — | 3.3 | — | ∘ |
| Example 14 | Alum | 1 | 99 | — | — | — | 3.6 | ± | ∘ |
| Example 15 | Sodium lauryl sulfate | 3 | 97 | — | — | — | 7.6 | — | ∘ |
| Example 16 | Lauryl sulfurate | 1 | 99 | — | — | — | 6.8 | — | ∘ |
| | triethanol amine | | | | | | | | |
| Example 17 | Sodium lauryl benzene | 3 | 97 | — | — | — | 7.2 | — | ∘ |
| | sulfonate | | | | | | | | |
| Example 18 | Sodium lauryl benzene | 0.3 | 99.7 | — | — | — | 7.2 | ± | ∘ |
| | sulfonate | | | | | | | | |
| Example 19 | Sodium polyoxyethylene | 3 | 97 | — | — | — | 4.7 | — | ∘ |
| | lauryl ether sulfate | | | | | | | | |
| Example 20 | Zinc sulfate.7H₂O | 3 | — | — | 97 | — | 5.4 | — | ∘ |
| Example 21 | Zinc sulfate.7H₂O | 0.3 | — | — | 99.7 | — | 5.7 | — | ∘ |
| Example 22 | Zinc sulfate.7H₂O | 0.03 | — | — | 99.97 | — | 6.7 | ± | ∘ |
| Example 23 | Zinc sulfate.7H₂O | 0.3 | — | — | — | 99.7 | 5.6 | ± | ∘ |
| Example 24 | Lead (II) acetate.2H₂O | 3 | — | — | 97 | — | 6.0 | — | ∘ |
| Example 25 | Lead (II) acetate.2H₂O | 0.3 | — | — | 99.7 | — | 6.0 | — | ∘ |
| Example 26 | Lead (II) acetate.2H₂O | 0.03 | — | — | 99.97 | — | 6.1 | — | ∘ |
| Example 27 | Lead (II) acetate.2H₂O | 0.01 | — | — | 99.99 | — | 6.2 | ± | ∘ |
| Example 28 | Lead (II) acetate.2H₂O | 0.3 | — | — | — | 99.7 | 5.9 | ± | ∘ |
| Comparative | — | — | 50 | 50 | — | — | 7.5 | ++ | ∘ |
| Example 1 | | | | | | | | | |
| Comparative | — | — | 100 | — | — | — | 7.0 | ++ | ∘ |
| Example 2 | | | | | | | | | |
| Comparative | — | — | — | — | 100 | — | 7.4 | ++ | ∘ |
| Example 3 | | | | | | | | | |
| Comparative | Tannic acid | 3 | 97 | — | — | — | 3.0 | ± | x |
| Example 4 | | | | | | | | | |
| Comparative | Succinic acid | 3 | 97 | — | — | — | 2.3 | ++ | ∘ |
| Example 5 | | | | | | | | | |
| Comparative | Aqueous sodium | 100 | — | — | — | — | 12.0 | ++ | ∘ |
| Example 6 | hydroxide solution | | | | | | | | |
| Comparative | Zinc sulfate.7H₂O | 3 | 97 | — | — | — | 5.1 | ++ | ∘ |
| Example 7 | | | | | | | | | |
| Comparative | Lead (II) acetate.2H₂O | 3 | 97 | — | — | — | 5.8 | ++ | ∘ |
| Example 8 | | | | | | | | | |

### Example 29

Disodium lauryl diphenyl ether disulfonate represented by the formula below was added as the divalent or more benzene sulfonate having a polyethylene chain in the molecule thereof to ion-exchanged water to prepare an allergen inhibitor solution. In the allergen inhibitor solution, the amount of disodium lauryl diphenyl ether disulfonate was 3% by weight and the amount of ion-exchanged water was 97% by weight.

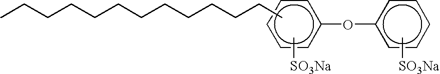

### Example 30

Disodium hexa(oxyethylene)diphenyl ether disulfonate represented by the formula below was added as the divalent or more benzene sulfonate having a polyoxyethylene chain in the molecule thereof to ion-exchanged water to prepare an allergen inhibitor solution. In the allergen inhibitor solution, the amount of disodium hexa(oxyethylene)diphenyl ether disulfonate was 3% by weight and the amount of ion-exchanged water was 97% by weight.

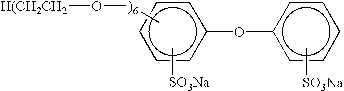

### Example 31

Disodium hexa(oxyethylene)lauryl diphenyl ether disulfonate represented by the formula below was added as the divalent or more benzene sulfonate having a polyoxyethylene chain and a polyethylene chain in the molecule thereof to ion-exchanged water to prepare an allergen inhibitor solution. In the allergen inhibitor solution, the amount of disodium hexa(oxyethylene)lauryl diphenyl ether disulfonate was 3% by weight and the amount of ion-exchanged water was 97% by weight.

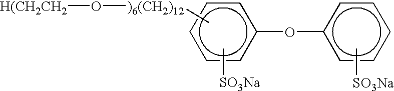

### Example 32

Tri(sodium lauryl sulfate)glycerin represented by the formula below was added as the divalent or more sulfate having a polyethylene chain in the molecule thereof to ion-exchanged water to prepare an allergen inhibitor solution. In the allergen inhibitor solution, the amount of tri(sodium lauryl sulfate)glycerin was 3% by weight and the amount of ion-exchanged water was 97% by weight.

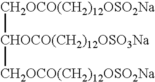

### Comparative Example 9

Ion-exchanged water was prepared.

### Reference Example 1

Sodium lauryl sulfate was added to ion-exchanged water to prepare an allergen inhibitor solution. In the allergen inhibitor solution, the amount of sodium lauryl sulfate was 3% by weight and the amount of ion-exchanged water was 97% by weight.

The allergen inhibitive action of each of the allergen inhibitors was measured by the above-described method, and foamability was measured by the following method, and the results are shown in Table 2.

Foamability

50 ml of the allergen inhibitor solution in each of Examples 29 to 32, Reference Example 1 and Comparative Example 9 or 50 ml ion-exchanged water in Comparative Example 9 was introduced into a 500-ml cylindrical beaker respectively. Each allergen inhibitor solution or ion-exchanged water in the beaker was foamed under stirring for 10 seconds at a stirring rate of 3000 rpm with a stirrer.

Just after stirring was finished or 1 minute after stirring, the surface of the allergen inhibitor solution in the beaker and the maximum height of foams on the surface were measured. Foamability was expressed in terms of the maximum height of foams.

TABLE 2

| | | Foamability (cm) | |
|---|---|---|---|
| | Allergen | Just after | After 1 |
| | inhibitive action | stirring | minute |
| Example 29 | − | 5 | 0 |
| Example 30 | ± | 8 | 0 |
| Example 31 | ± | 9 | 0 |
| Example 32 | − | 3 | 1 |
| Comparative Example 9 | ++ | 1 | 0 |
| Reference Example 1 | − | 12 | 8 |

### Examples 33 to 37

Sodium sulfate anhydride (Ishida Chemical Industries Co., Ltd.), potassium sulfate (Chisso Corporation), ammonium sulfate, and aluminum sulfate anhydride (Taimei Chemical Co., Ltd.) were dissolved in the amounts shown in Table 3 in ion-exchanged water to prepare allergen inhibitor solutions. The resulting allergen inhibitor solution was introduced into a trigger-type spray container (about 0.8 ml spray by spraying once).

### Examples 38 to 40

Aluminum sulfate anhydride (Taimei Chemical Co., Ltd.), sodium sulfate anhydride (Ishida Chemical Industries Co., Ltd.), calcined alum (aluminum potassium sulfate (food additive) manufactured by Wako Pure Chemical Industries, Ltd.) and tannic acid were dissolved in the amounts shown in Table 3 in ion-exchanged water to prepare allergen inhibitor solutions. The resulting allergen inhibitor solution was introduced into a trigger-type spray container (about 0.8 ml spray by spraying once).

### Comparative Example 10

Tannic acid was dissolved in the amount shown in Table 3 in ion-exchanged water to prepare an allergen inhibitor solution. The resulting allergen inhibitor solution was introduced into a trigger-type spray container (about 0.8 ml spray by spraying once).

The allergen inhibitive action, repeatability, surface properties, coloration and stability of the allergen inhibitor solutions were measured by the following methods, and the results are shown in Table 3.

Allergen Inhibitive Action

A new carpet was sprayed in an amount of 50 μg/m² with acarian antigen-containing dust (acarian antigen was quantified by Mighty Checker™ manufactured by Shinto Fine Co., Ltd.), and then the bottom of the carpet was vibrated with a vibrator to penetrate the dust into the carpet, whereby a polluted carpet was prepared.

The upper surface of the polluted carpet was sprayed 4 times with the allergen inhibitor solution from the spray-type container. After the carpet was left for 24 hours, the amount of allergens remaining on the polluted carpet was measured with an allergen measuring device (Dani Scan™ manufactured by Asahi Beer Pharmaceutical Co., Ltd.) and judged according to the following criteria.

Criteria
1: Not contaminated with acarian allergen (T=0).
2: Slightly contaminated with acarian allergen (T<C).
3: Contaminated with acarian allergen (T=C).
4: Extremely contaminated with acarian allergen (T>C).
   T indicates the test line, and C indicates the control line.

Repeatability

The upper surface of a polluted carpet was sprayed 4 times with the allergen inhibitor solution from the spray-type container. The polluted carpet was prepared in the same manner as in examination of the allergen inhibitive action described above. This operation of spraying was conducted 3 times.

Thereafter, the amount of allergens remaining on the polluted carpet was measured with an allergen measuring device (Dani Scan™ manufactured by Asahi Beer Pharmaceutical Co., Ltd.) and judged according to the same criteria as in examination of the allergen inhibitive action described above.

Surface Properties

After the allergen inhibitive action was measured, whether the surface of the polluted carpet was rigidly denatured or not was judged by touching. ◯ was given when there was no problem, and x was given when there was a problem.

Coloration

The upper surface of a new white carpet was uniformly sprayed 4 times with the allergen inhibitor solution, and whether the carpet was colored or not was visually observed. ◯ was given when no coloration was observed, and x was given when coloration was recognized.

Stability

After the allergen inhibitor solution was left at 2° C. for 24 hours, whether the solute in the allergen inhibitor solution was recrystallized or not was visually observed. ◯ was given when the solute was not recrystallized, and x was given when the solute was recrystallized.

TABLE 3
(% by weight)

| | | | | | | | | | Com- |
|---|---|---|---|---|---|---|---|---|---|
| | | | | | | | | | parative |
| | Example | | | | | | | | Example |
| | 33 | 34 | 35 | 36 | 37 | 38 | 39 | 40 | 10 |
| Sodium sulfate | 1 | 2 | 3 | — | — | — | 17 | 0.2 | — |
| anhydride | | | | | | | | | |
| Potassium sulfate | — | — | — | 3 | 3 | — | — | — | — |
| Ammonium sulfate | — | — | — | — | — | — | — | — | — |
| Aluminum sulfate | 1 | 2 | 3 | 3 | 3 | — | 35 | 0.2 | — |
| anhydride | | | | | | | | | |
| Calcined alum | — | — | — | — | — | 5 | — | — | — |
| Tannic acid | — | — | — | — | — | — | — | — | 3 |
| Ion-exchanged | 98 | 96 | 94 | 94 | 94 | 95 | 48 | 99.6 | 97 |
| water | | | | | | | | | |
| Allergen inhibitive | 3 | 2 | 2 | 2 | 2 | 1 | 1 | 4 | 2 |
| action | | | | | | | | | |
| Repeatability | 1 | 1 | 1 | 1 | 1 | 1 | 1 | 4 | 1 |
| Surface properties | ∘ | ∘ | ∘ | ∘ | ∘ | x | x | ∘ | ∘ |
| Coloration | ∘ | ∘ | ∘ | ∘ | ∘ | ∘ | ∘ | ∘ | x |
| Stability | ∘ | ∘ | ∘ | ∘ | ∘ | x | x | ∘ | ∘ |

### Example 41

100 parts by weight of 4-vinyl phenol (10% by weight purity in propylene glycol, manufactured by Lancaster Co.) were added to an aqueous emulsified dispersion consisting of 1 part by weight of benzoyl peroxide (first grade reagent of 75% purity, manufactured by Sigma Aldrich Co. Ltd.), 1 part by weight of sodium lauryl sulfate (Emal 2F Needle™, 90% by weight active ingredient or solids content, manufactured by Kao Corporation), 10 parts by weight of chlorobenzene (special grade reagent of 99.5% purity, manufactured by Sigma Aldrich Co. Ltd.) and 1000 parts by weight of ion-exchanged water to prepare a fiber treatment solution.

20 parts by weight of a fabric (100 g/m²) consisting of polyethylene terephthalate fibers were dipped in the fiber treatment solution and subjected to graft polymerization by heating at 100° C. for 60 minutes. The fabric was dipped in ion-exchanged water at 100° C. for 30 minutes, and then neutralized with 0.5% by weight aqueous sodium carbonate at 50° C. for 30 minutes. Then, the fabric was washed with water and dried to give a fiber product consisting of allergen-inhibiting fibers.

### Example 42

2 parts by weight of polytyrosine (weight-average molecular weight (Mw) of 18000 to 36000, manufactured by INC Biochemicals, Inc.), 2 parts by weight of ethyl acrylate-methyl methacrylate copolymer as a binder (Eudoragit NE30D™, solids content of 30% by weight, manufactured by Rohm Pharma), 0.3 part by weight of a nonionic surfactant (Emulgen 911™ manufactured by Kao Corporation) and 100 parts by weight of ion-exchanged water as a solvent were mixed and stirred to prepare a fiber treatment solution.

The surface of a nonwoven fabric (100 g/m²) consisting of polyester fibers was sprayed uniformly in an amount of 20 μl/cm² with the fiber treatment solution and dried by leaving it at room temperature for 8 hours to give a fiber product consisting of allergen-inhibiting fibers.

### Example 43

10 parts by weight of aluminum potassium sulfate (first grade, manufactured by Wako Pure Chemical Industries, Ltd.) were dissolved in 45 parts by weight of ethyl alcohol (first grade, manufactured by Nakarai Tesque) and 45 parts by weight of ion-exchanged water as a solvent to prepare a fiber treatment solution.

The surface of a nonwoven fabric (100 g/m²) consisting of polyester fibers was sprayed uniformly in an amount of 10 μl/cm² with the fiber treatment solution and then dried by leaving it a room temperature for 8 hours to give a fiber product consisting of allergen-inhibiting fibers.

### Comparative Example 11

A fabric (100 g/m²) consisting of polyethylene terephthalate fibers was used without impregnation of the fabric with any allergen inhibitor.

### Comparative Example 12

A nonwoven fabric (100 g/m²) consisting of polyester fibers was used without impregnation of the fabric with any allergen inhibitor.

The fiber products, the fabrics and the nonwoven fabrics used in the Comparative Examples were measured for their allergen inhibitive action by methods described below, and the results are shown in Table 4.

Allergen Inhibitive Action 1

10 g test fabric specimen (flat rectangle of 33 cm in length×30 cm in width) was obtained from each of the fiber products, the fabrics and the nonwoven fabrics. 5 g dust (allergen content: 2 mg/g) was dispersed in a mixed solvent of 50 g ethyl alcohol and 50 g ion-exchanged water, to form an allergen preparation. 1 ml of the allergen preparation was sprayed onto the surface of the test fabric specimen.

The test fabric specimen was left at room temperature for 8 hours, and the amount of allergens in the test fabric specimen was measured with an allergen measuring device (Dani Scan™ manufactured by Asahi Beer Pharmaceutical Co., Ltd.) and judged according to the following criteria.

Criteria
1: Not contaminated with acarian allergen (T=0).
2: Slightly contaminated with acarian allergen (T<C).
3: Contaminated with acarian allergen (T=C).
4: Extremely contaminated with acarian allergen (T>C).
   T indicates the test line, and C indicates the control line.

Allergen Inhibitive Action 2

10 g test fabric specimen (flat rectangle of 33 cm in length×30 cm in width) was obtained from each of the fiber products, the fabrics and the nonwoven fabrics. 5 g dust (allergen content: 2 mg/g) was dispersed in a mixed solvent of 50 g ethyl alcohol and 50 g ion-exchanged water, to form an allergen preparation. 1 ml of the allergen preparation was sprayed onto the surface of the test fabric specimen.

After the test fabric specimen was left at room temperature for 2 hours, allergen components were extracted from the test fabric specimen by an allergen extractor (Mighty Checker™ manufactured by Shinto Fine Co., Ltd.), and the amount of allergens was determined. The results are shown in Table 4.

Judgment Criteria
++: Acarian allergen level>35 μg/m²
+: Acarian allergen level 10 μg/m²
±: Acarian allergen level 5 μg/m²

−: Acarian allergen level<1 μg/m²

TABLE 4

| | Allergen inhibitive | Allergen inhibitive |
|---|---|---|
| | action 1 | action 2 |
| Example 41 | 1 | − |
| Example 42 | 2 | ± |
| Example 43 | 1 | − |
| Comparative | 4 | ++ |
| Example 11 | | |
| Comparative | 4 | ++ |
| Example 12 | | |

### Example 44

A resin composition consisting of 100 parts by weigh of polyethylene terephthalate (limiting viscosity [η]=0.65) and 100 parts by weight of poly-4-vinyl phenol (Maruka Linker M™, weight-average molecular weight (Mw) of 5500, manufactured by Maruzen Petrochemical Co., Ltd.), then fed to a press kneader and kneaded at 260° C. for 20 minutes.

The resin composition kneaded in the manner described above was fed to a single screw extruder, melt-kneaded and extruded into a bar which was then cut into pellets of predetermined length. The pellets were spun by melt spinning (spinning pack filter: 270 mesh size), stretched, washed with water and dried to give allergen-inhibiting fibers.

### Comparative Example 13

Polyethylene terephthalate (limiting viscosity [η]=0.65) was fed to a single screw extruder, melt-kneaded and extruded into a bar which was then cut into pellets of predetermined length. The pellets were spun by melt spinning (spinning pack filter: 270 mesh size), stretched, washed with water and dried to give polyethylene terephthalate fibers.

The allergen-inhibiting fibers and the polyethylene terephthalate fibers were measured for their allergen inhibitive action by the following method. The results are shown in Table 5.

Allergen Inhibitive Actions 1 and 2

Allergen inhibitive actions 1 and 2 were measured in the same manner as in Example 41 except that 10 g of the allergen-inhibiting fibers or the polyethylene terephthalate fibers were used in place of the test fabric specimen.

TABLE 5

| | Allergen inhibitive | Allergen inhibitive |
|---|---|---|
| | action 1 | action 2 |
| Example 44 | 1 | − |
| Comparative | 4 | ++ |
| Example 13 | | |

### Example 45

100 parts by weight of 4-vinyl phenol (10% by weight purity in propylene glycol, manufactured by Lancaster Co.) and 20 parts by weight of polyethylene glycol as a hygroscopic compound (weight-average molecular weight of 7500, manufactured by Wako Pure Chemical Industries, Ltd.) were added to an aqueous emulsified dispersion consisting of 1 part by weight of benzoyl peroxide (first grade reagent of 75% purity, manufactured by Sigma Aldrich Co. Ltd.), 1 part by weight of sodium lauryl sulfate (Emal 2F Needle™, 90% by weight active ingredient or solids content, manufactured by Kao Corporation), 10 parts by weight of chlorobenzene (special grade reagent of 99.5% purity, manufactured by Sigma Aldrich Co. Ltd.) and 1000 parts by weight of ion-exchanged water to prepare a fiber treatment solution.

20 parts by weight of a fabric consisting of polyethylene terephthalate fibers was dipped in the fiber treatment solution and subjected to graft polymerization by heating at 100% for 60 minutes. The fabric was dipped in ion-exchanged water at 100° C. for 30 minutes, and then neutralized with 0.5% by weight aqueous sodium carbonate at 50° C. for 30 minutes. Then, the fabric was washed with water and dried to give a fiber product consisting of allergen-inhibiting fibers.

Ion-exchanged water was dropped onto the surface of the fiber product and then left for 15 minutes to moisten the surface sufficiently, and the pH of the surface of the fiber product was measured by a pH test paper, indicating that the pH was 7.0.

### Example 46

2 parts by weight of polytyrosine as an allergen inhibitor (weight-average molecular weight of 18000 to 36000, manufactured by INC Biochemicals, Inc.), 2 parts by weight of polyethylene glycol as a hygroscopic compound (weight-average molecular weight of 7500, manufactured by Wako Pure Chemical Industries, Ltd.), 2 parts by weight of ethyl acrylate-methyl methacrylate copolymer as a binder (Eudoragit NE30D™, solids content of 30% by weight, manufactured by Rohm Pharma), 0.3 part by weight of a nonionic surfactant (Emulgen 911™ manufactured by Kao Corporation), 100 parts by weight of a solvent ion-exchanged water and 0.1 part by weight of barium hydroxide (Wako Pure Chemical Industries, Ltd.) were mixed and stirred to prepare a fiber treatment solution.

The surface of a nonwoven polyester fabric (100 g/m²) was sprayed uniformly in an amount of 20 μl/cm² with the fiber treatment solution and then dried by leaving it a room temperature for 8 hours, to give a fiber product consisting of allergen-inhibiting fibers.

Ion-exchanged water was dropped onto the surface of the fiber product and then left for 15 minutes to moisten the surface sufficiently, and the pH of the surface of the fiber product was measured by a pH test paper, indicating that the pH was 8.3.

### Example 47

A resin composition consisting of 100 parts by weigh of polyethylene terephthalate (limiting viscosity [α]=0.65), 20 parts by weight of poly-4-vinyl phenol as an allergen inhibitor (Maruka Linker M™, weight-average molecular weight of 5500, manufactured by Maruzen Petrochemical Co., Ltd.), 10 parts by weight of polypropylene glycol as a hygroscopic compound (diol form, weight-average molecular weight of 3000, manufactured by Wako Pure Chemical Industries, Ltd.) and 10 parts by weight of magnesium hydroxide (Wako Pure Chemical Industries, Ltd.) was kneaded with a press kneader at 260° C. for 20 minutes.

Then, the resin composition kneaded in the manner described above was fed to a single screw extruder, melt-kneaded and extruded into a bar which was then cut into pellets of predetermined length. The pellets were spun by melt spinning (spinning pack filter: 270 mesh size), stretched, washed with water and dried to give allergen-inhibiting fibers. The allergen-inhibiting fibers were plain-weaved to give a fabric (fiber product) consisting of the allergen-inhibiting fibers.

Ion-exchanged water was dropped onto the surface of the fiber product and then left for 15 minutes to moisten the surface sufficiently, and the pH of the surface of the fiber product was measured by a pH test paper, indicating that the pH was 11.0.

### Example 48

100 parts by weight of 4-vinyl phenol (10% by weight purity in propylene glycol solution, manufactured by Lancaster Co.) and 20 parts by weight of polyethylene glycol as a hygroscopic compound (weight-average molecular weight of 7500, manufactured by Wako Pure Chemical Industries, Ltd.) were added to an aqueous emulsified dispersion consisting of 1 part by weight of benzoyl peroxide (first grade reagent of 75% purity, manufactured by Sigma Aldrich Co. Ltd.), 1 part by weight of sodium lauryl sulfate (Emal 2F Needle™, 90% by weight active ingredient or solids content, manufactured by Kao Corporation), 10 parts by weight of chlorobenzene (special grade reagent of 99.5% purity, manufactured by Sigma Aldrich Co. Ltd.) and 1000 parts by weight of ion-exchanged water to prepare a fiber treatment solution.

20 parts by weight of a fabric consisting of polyethylene terephthalate fibers was dipped in the fiber treatment solution and subjected to graft polymerization by heating at 100° C. for 60 minutes. The fabric was dipped in ion-exchanged water at 100° C. for 30 minutes, and then treated with 0.1 N hydrochloric acid at 50° C. for 30 minutes to acidify the surface of the fiber product, then washed with water and dried to give a fiber product consisting of allergen-inhibiting fibers.

Ion-exchanged water was dropped onto the surface of the fiber product and then left for 15 minutes to moisten the surface sufficiently, and the pH of the surface of the fiber product was measured by a pH test paper, indicating that the pH was 3.0.

### Example 49

2 parts by weight of polytyrosine as an allergen inhibitor (weight-average molecular weight of 18000 to 36000, manufactured by INC Biochemicals, Inc.), 2 parts by weight of polyethylene glycol as a hygroscopic compound (weight-average molecular weight of 7500, manufactured by Wako Pure Chemical Industries, Ltd.), 2 parts by weight of ethyl acrylate-methyl methacrylate copolymer as a binder (Eudoragit NE30D™, solids content of 30% by weight, manufactured by Rohm Pharma), 0.3 part by weight of a nonionic surfactant (Emulgen 911™ manufactured by Kao Corporation), 100 parts by weight of ion-exchanged water as a solvent and 0.1 part by weight of 0.01 N sulfuric acid (Wako Pure Chemical Industries, Ltd.) were mixed and stirred to prepare a fiber treatment solution.

A nonwoven fabric (100 g/m²) consisting of polyester fibers was sprayed uniformly in an amount of 20 μl/cm² with the fiber treatment solution and dried by leaving it at room temperature for 8 hours to give a fiber product consisting of allergen-inhibiting fibers.

Ion-exchanged water was dropped onto the surface of the fiber product and then left for 15 minutes to moisten the surface sufficiently, and the pH of the surface of the fiber product was measured by a pH test paper, indicating that the pH was 3.3.

### Example 50

A resin composition consisting of 100 parts by weigh of polyethylene terephthalate (limiting viscosity [η]=0.65), 20 parts by weight of poly-4-vinyl phenol as an allergen inhibitor (Maruka Linker M™, weight-average molecular weight of 5500, manufactured by Maruzen Petrochemical Co., Ltd.), 10 parts by weight of polypropylene glycol as a hygroscopic compound (diol form, weight-average molecular weight of 3000, manufactured by Wako Pure Chemical Industries, Ltd.) and 1 part by weight of iron(III)chloride (Wako Pure Chemical Industries, Ltd.) was fed to a press kneader and kneaded at 260° C. for 20 minutes.

The resin composition kneaded in the manner described above was fed to a single screw extruder, melt-kneaded and extruded into a bar which was then cut into pellets of predetermined length. The pellets were spun by melt spinning (spinning pack filter: 270 mesh size), stretched, washed with water and dried to give allergen-inhibiting fibers. The allergen-inhibiting fibers were plain-weaved to give a fabric (fiber product) consisting of the allergen-inhibiting fibers.

Ion-exchanged water was dropped onto the surface of the fiber product and then left for 15 minutes to moisten the surface sufficiently, and the pH of the surface of the fiber product was measured by a pH test paper, indicating that the pH was 2.7.

### Comparative Example 14

A fabric (fiber product) consisting of polyethylene terephthalate fibers was used as such without incorporating any allergen inhibitor.

### Comparative Example 15

A nonwoven fabric (fiber product) consisting of polyester fibers was used as such without incorporating any allergen inhibitor.

Ion-exchanged water was dropped onto the surface of the fiber product and then left for 15 minutes to moisten the surface sufficiently, and the pH of the surface of the fiber product was measured by a pH test paper, indicating that the pH was 7.5.

### Comparative Example 16

Polyethylene terephthalate (limiting viscosity [η]=0.65) was fed to a single screw extruder, melt-kneaded and extruded into a bar which was then cut into pellets of predetermined length. The pellets were spun by melt spinning (spinning pack filter: 270 mesh size), stretched, washed with water and dried to give polyethylene terephthalate fibers. The polyethylene terephthalate fibers were plain-weaved to give a fabric (fiber product).

Ion-exchanged water was dropped onto the surface of the fiber product and then left for 15 minutes to moisten the surface sufficiently, and the pH of the surface of the fiber product was measured by a pH test paper, indicating that the pH was 6.7.

The fiber product was measured for allergen inhibitive action by the following method. The results are shown in Table 6.

Allergen Inhibitive Action

Each fiber product was cut into 20 flat square test fabric specimens having a 10-cm side. Dust (containing 10 μg/g Der p1 allergen) was dispersed in a mixed solution of 90 g ethyl alcohol and 10 g ion-exchanged water, to prepare an allergen solution.

Each of the test fabric specimens was sprayed with 5 ml allergen solution and then dried in an oven at 50° C. for 5 minutes, whereby 20 polluted fabric specimens were prepared. The amount of allergens in the polluted fabric specimens just after preparation, and the amount of allergens in the polluted fabric specimens after being left for 12 hours at 25° C. in 75% RH (absolute humidity 17.4 g/m³) in a thermostatic humidistat, were measured by a method described below. Out of the 20 polluted fabric specimens, 10 polluted fabric specimens were used to measure the amount of allergens just after preparation and the other 10 polluted fabric specimens were used to measure the amount of allergens after being left for 12 hours. The amount of allergens shown in the table is the average amount of allergens in the 10 polluted fabric specimens.

First, the polluted fabric specimen was crumpled and placed in a 15 ml glass test tube, and 10 ml extracting buffer (phosphate buffer, pH 7.35, containing 1% by weight of BSA and 0.05% by weight of polyoxyethylene (20) sorbitan monolaurate) was introduced into the glass test tube.

Thereafter, the glass test tube was shaken enough for 20 minutes and immediately after that an extract from the polluted fabric specimen was recovered. The amount of allergens in the resulting extract was measured by an ELISA kit (LCD Allergy Institute) and determined in terms of the amount of Der p 1/m².

TABLE 6

| | Just after preparation | After left for 12 hours |
|---|---|---|
| | (ng/m²) | (ng/m²) |
| Example 45 | 2537 (SD = 220) | 359 (SD = 78) |
| Example 46 | 3752 (SD = 311) | 153 (SD = 56) |
| Example 47 | 2841 (SD = 199) | 118 (SD = 32) |
| Example 48 | 2911 (SD = 201) | 1520 (SD = 115) |
| Example 49 | 3007 (SD = 228) | 1734 (SD = 98) |
| Example 50 | 3589 (SD = 219) | 1890 (SD = 333) |
| Comparative | 2509 (SD = 250) | 2733 (SD = 149) |
| Example 14 | | |
| Comparative | 2948 (SD = 329) | 2359 (SD = 205) |
| Example 15 | | |
| Comparative | 3589 (SD = 219) | 2751 (SD = 276) |
| Example 16 | | |

### Example 51

2.4 g nonwoven fabric (KP8340, manufactured by Sanshoshigyo Co., Ltd.) in a flat rectangular form (length 30 cm, width 20 cm) was impregnated with 3.6 g allergen inhibitor solution prepared by dispersing 1% by weight of sodium polyoxyethylene lauryl ether sulfate (Kao Corporation) as an allergen inhibitor and 20% by weight of propylene glycol as an oily substance-solubilizing solvent in 79% by weight of ion-exchanged water, and the nonwoven fabric was left in a sealed vessel for 24 hours to give an allergen-inhibiting sheet.

### Example 52

2.4 g nonwoven fabric (KP8340, manufactured by Sanshoshigyo Co., Ltd.) in a flat rectangular form (length 30 cm, width 20 cm) was impregnated with 6 g allergen inhibitor solution prepared by dispersing 0.5% by weight of tannic acid (Wako Pure Chemical Industries, Ltd.) as an allergen inhibitor and 9.5% by weight of glycerin as an oily substance-solubilizing solvent in 90% by weight of ion-exchanged water, and the nonwoven fabric was left in a sealed vessel for 24 hours to give an allergen-inhibiting sheet.

### Example 53

4.1 g cotton fabric in a flat rectangular form (length 15 cm, width 20 cm) was impregnated with 3 g allergen inhibitor solution prepared by dispersing 5% by weight of alum (aluminum potassium sulfate manufactured by Wako Pure Chemical Industries, Ltd.) as an allergen inhibitor in 95% by weight of ion-exchanged water, and the cotton fabric was left in a sealed vessel for 24 hours to give an allergen-inhibiting sheet.

### Example 54

4.1 g cotton fabric in a flat rectangular form (length 15 cm, width 20 cm) was impregnated with 3 g allergen inhibitor solution prepared by dispersing 3% by weight of sodium lauryl sulfate (Kao Corporation) as an allergen inhibitor and 40% by weight of propylene glycol as an oily substance-solubilizing solvent in 57% by weight of ion-exchanged water, and the cotton fabric was left in a sealed vessel for 24 hours to give an allergen-inhibiting sheet.

### Example 55

2.7 g nonwoven fabric (#281, manufactured by Kureha Teck Co., Ltd.) in a flat rectangular form (length 15 cm, width 20 cm) was impregnated with 3 g allergen inhibitor solution prepared by dissolving 3% by weight of poly-4-vinyl phenol (weigh-average molecular weight 8000, manufactured by Aldrich) as an allergen inhibitor and 20% by weight of propylene glycol as an oily substance-solubilizing solvent in a mixed solvent of 30% by weight of ion-exchanged water and 47% by weight of ethyl alcohol, and the nonwoven fabric was left in a sealed vessel for 24 hours to give an allergen-inhibiting sheet.

### Example 56

3% by weight of zinc sulfate.7H₂O (Wako Pure Chemical Industries, Ltd.) was dissolved in 97% by weight of a phosphate buffer (pH 7.35) adjusted to a concentration of 0.01 M prepared by dissolving 0.01 M monosodium phosphate.2H₂O (Kanto Kagaku) and 0.01 M disodium phosphate.12H₂O (Wako Pure Chemical Industries, Ltd.) as phosphates in ion-exchanged water, to give an allergen inhibitor solution. 2.7 g nonwoven fabric (#281, manufactured by Kureha Teck Co., Ltd.) in a flat rectangular form (length 15 cm, width 20 cm) was impregnated with 3 g allergen inhibitor solution and then left in a sealed vessel for 24 hours to give an allergen-inhibiting sheet.

### Comparative Example 17

2.7 g nonwoven fabric (#281, manufactured by Kureha Teck Co., Ltd.) in a flat rectangular form (length 15 cm, width 20 cm) was impregnated with a solution prepared by dissolving 20% by weight of propylene glycol as an oily substance-solubilizing solvent in 80% by weight of ion-exchanged water, and then left in a sealed vessel for 24 hours to give a cleaning sheet.

### Reference Example 2

6 g allergen inhibitor solution prepared by dissolving 1% by weight of sodium polyoxyethylene lauryl ether sulfate (Kao Corporation) as an allergen inhibitor and 20% by weight of propylene glycol as an oily substance-solubilizing solvent in 79% by weight of ion-exchanged water was introduced into a trigger-type spray container (about 0.8 ml spray by spraying once).

The allergen-inhibiting sheets, the allergen inhibitor solutions and the cleaning sheet were measured for their allergen inhibitive action and surface properties by methods described below. The results are shown in Table 7.

Allergen Inhibitive Action

The allergen inhibitor sheet and the cleaning sheet were used to clean 4 places, that is, tatami mats in a living room in family T, tatami mats in a bed room in family S, tatami mats in a bed room in family M and tatami mats in a living room in family N, by wiping for 90 seconds/tatami mat.

The allergen inhibitor solution in Reference Example 2 was uniformly sprayed twice on tatami mats, and then the tatami mats were cleaned by spreading the allergen inhibitor uniformly with an acrylic plate on the surface of the tatami mats. The amount of allergens in the tatami mats before cleaning was measured in Reference Example 3.

After cleaning, the amount of allergens in a central portion (290 mm×210 mm) in the cleaned region was measured with an allergen measuring device (Dani Scan™ manufactured by Asahi Beer Pharmaceutical Co., Ltd.) and judged according to the following criteria.

Criteria
1: Not contaminated with acarian allergen (T=0).
2: Slightly contaminated with acarian allergen (T<C).
3: Contaminated with acarian allergen (T=C).
4: Extremely contaminated with acarian allergen (T>C).
   T indicates the test line, and C indicates the control line.

Surface Properties

The allergen-inhibiting sheet and the cleaning sheet were used to wipe the surface of unused tatami mats 3 times, and then the surface shape of the tatami mats was visually observed. The allergen inhibitor solution in Reference Example 2 was sprayed twice onto the surface of tatami mats and then wiped off, and the surface shape of the tatami mats was visually observed according to the following criteria:
◯ No deformation.
Δ A few waves on the surface of tatami mats.

x A large number of waves on the surface of tatami mats.

TABLE 7

| | Allergen inhibitive action | | | | Surface |
|---|---|---|---|---|---|
| | Family T | Family S | Family M | Family N | properties |
| Example 51 | 1 | 1 | 2 | 1 | ∘ |
| Example 52 | 1 | 2 | 2 | 1 | Δ |
| Example 53 | 1 | 1 | 1 | 1 | ∘ |
| Example 54 | 1 | 1 | 1 | 1 | ∘ |
| Example 55 | 1 | 1 | 1 | 1 | ∘ |
| Example 56 | 1 | 1 | 1 | 1 | ∘ |
| Comparative | 3 | 3 | 4 | 3 | Δ |
| Example 17 | | | | | |
| Reference | 2 | 3 | 3 | 3 | x |
| Example 2 | | | | | |
| Reference | 3 | 4 | 4 | 4 | — |
| Example 3 | | | | | |

1. An allergen inhibitor comprising at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof.

2. The allergen inhibitor according to claim 1, wherein the aromatic hydroxy compound is a compound having, in a linear polymer, at least one of substituent groups represented by the general formulas (1) to (6): 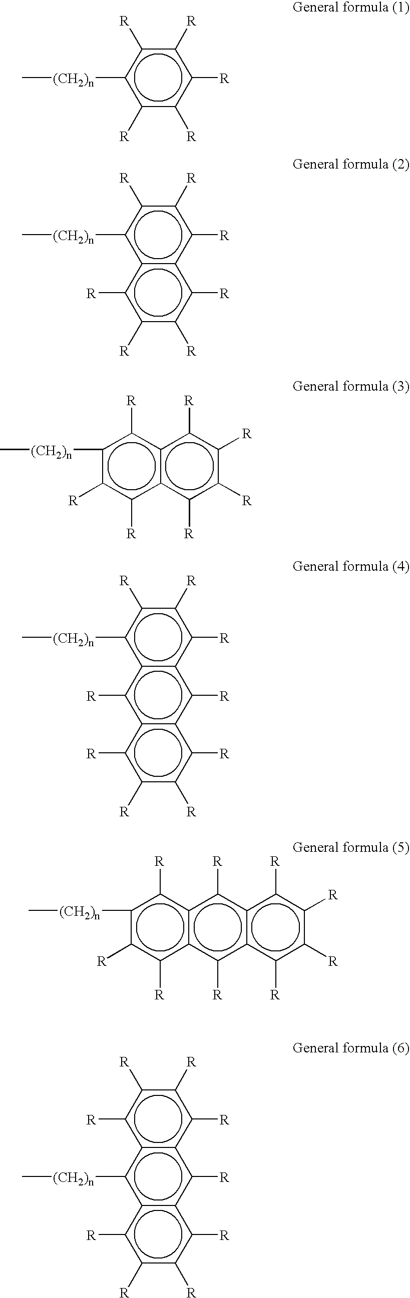
wherein R is a hydrogen atom or a hydroxyl group, and at least one R is a hydroxyl group, and n is an integer of 0 to 5.

3. The allergen inhibitor according to claim 2, wherein only one of the substituent groups R is a hydroxyl group, and all the other substituent groups R are hydrogen atoms.

4. The allergen inhibitor according to claim 1, wherein the aromatic hydroxy compound is obtained by polymerizing or copolymerizing a monomer having at least one of substituent groups represented by the general formulas (1) to (6): 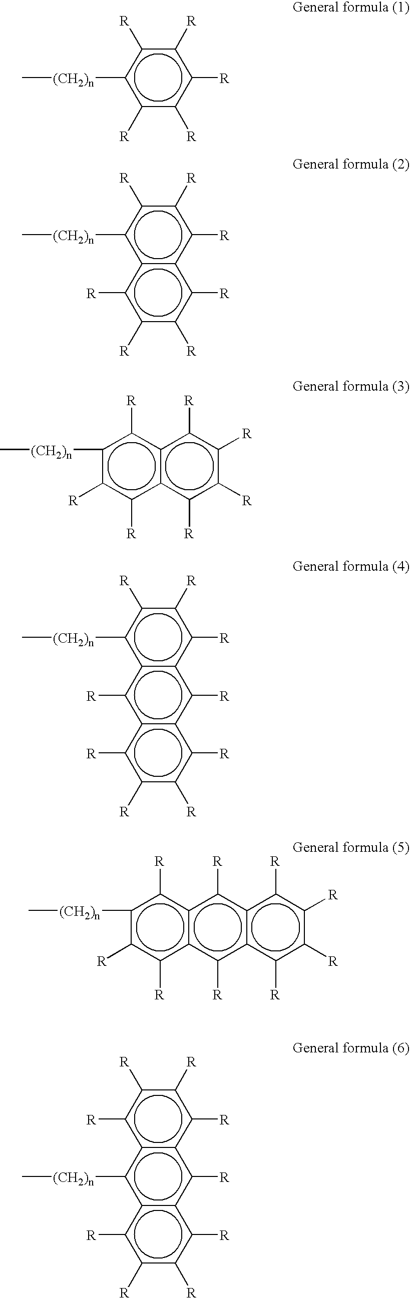
wherein R is a hydrogen atom or a hydroxyl group, and at least one R is a hydroxyl group, and n is an integer of 0 to 5.

5. The allergen inhibitor according to claim 4, wherein only one of the substituent groups R is a hydroxyl group, and all the other substituent groups R are hydrogen atoms.

6. The allergen inhibitor according to claim 1, wherein the aromatic hydroxy compound is an aromatic heterocyclic hydroxy compound.

7. The allergen inhibitor according to claim 1, wherein the divalent or more sulfate having either or both of a polyoxyethylene chain and a polyethylene chain in the molecule thereof is alkyl diphenyl ether disulfonate.

8. An allergen inhibitor comprising a phosphate and either or both of zinc sulfate and lead acetate.

9. An allergen inhibitor comprising an aqueous solution containing aluminum sulfate and at least one sulfate selected from the group consisting of sodium sulfate, potassium sulfate, ammonium sulfate and thallium sulfate.

10. The allergen inhibitor according to claim 9, which comprises sodium sulfate and aluminum sulfate, wherein the total concentration of sodium sulfate and aluminum sulfate is 0.5 to 50% by weight.

11. A method of inhibiting allergens which comprises supplying at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof, in an object where allergens exist to inhibit the allergens.

12. The method of inhibiting allergens according to claim 11, wherein the aromatic hydroxy compound is a compound having, in a linear polymer, at least one of substituent groups represented by the general formulas (1) to (6): 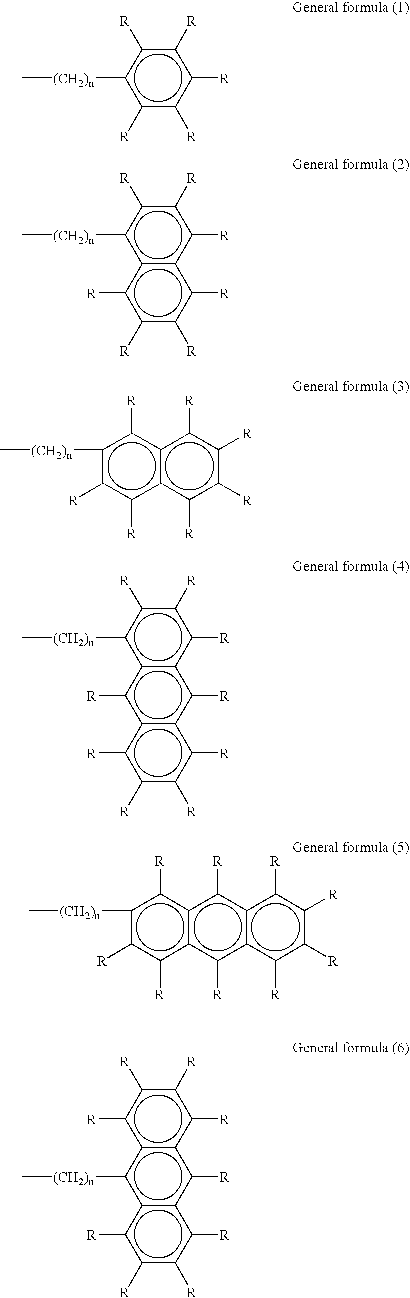
wherein R is a hydrogen atom or a hydroxyl group, and at least one R is a hydroxyl group, and n is an integer of 0 to 5.

13. The method of inhibiting allergens according to claim 11, wherein the aromatic hydroxy compound is obtained by polymerizing or copolymerizing a monomer having at least one of substituent groups represented by the general formulas (1) to (6): 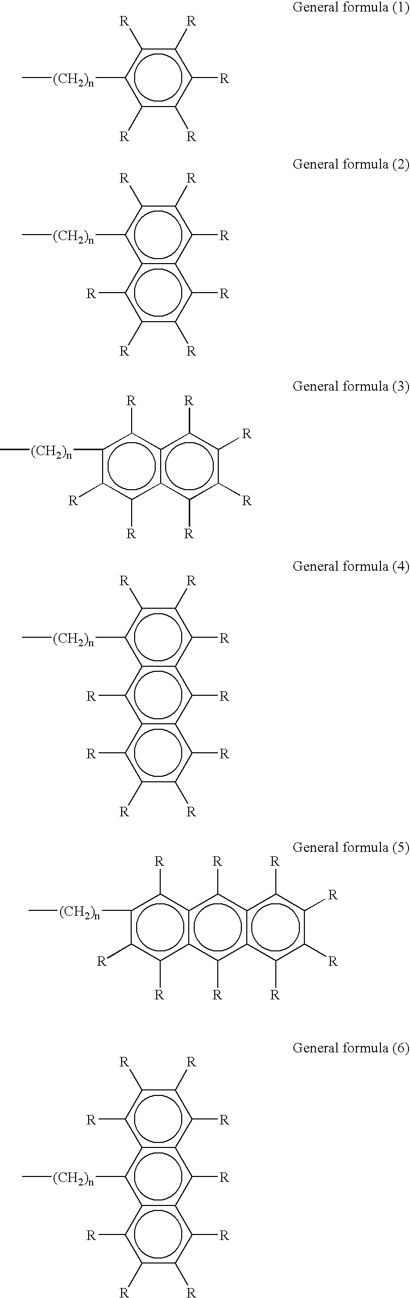
wherein R is a hydrogen atom or a hydroxyl group, and at least one R is a hydroxyl group, and n is an integer of 0 to 5.

14. A method of inhibiting allergens which comprises supplying an aqueous solution containing aluminum sulfate and at least one sulfate selected from the group consisting of sodium sulfate, potassium sulfate, ammonium sulfate and thallium sulfate, in an object where allergens exist to inhibit the allergens.

15. Allergen-inhibiting fibers comprising an allergen inhibitor contained in fibers.

16. The allergen-inhibiting fibers according to claim 15, wherein the allergen inhibitor is chemically bound to fibers.

17. The allergen-inhibiting fibers according to claim 15, wherein the allergen inhibitor is chemically bound to fibers by graft reaction.

18. The allergen-inhibiting fibers according to claim 15, wherein the allergen inhibitor is physically fixed or mixed in fibers.

19. The allergen-inhibiting fibers according to claim 15, wherein the allergen inhibitor is an aromatic hydroxy compound.

20. The allergen-inhibiting fibers according to claim 19, wherein the aromatic hydroxy compound is a compound having, in a linear polymer, at least one of substituent groups represented by the general formulas (1) to (6): 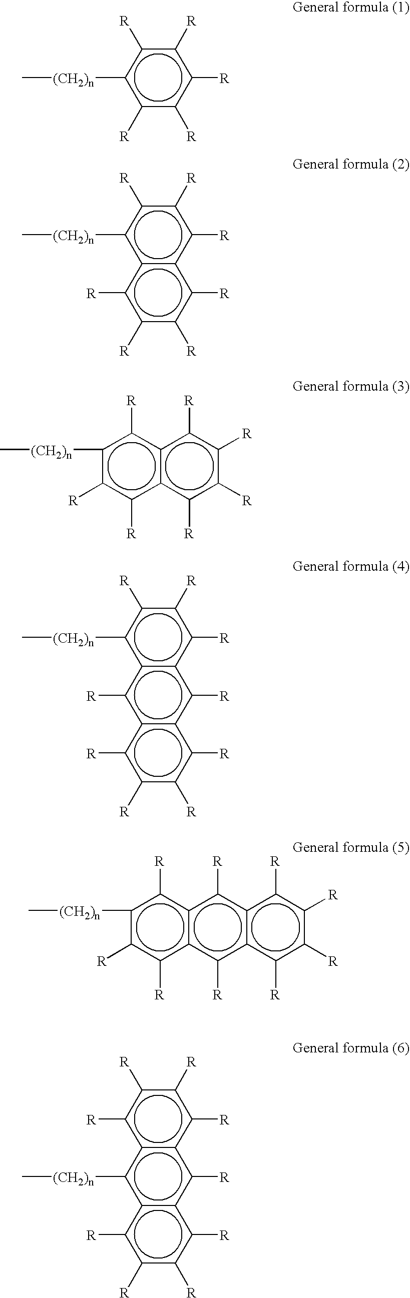
wherein R is a hydrogen atom or a hydroxyl group, and at least one R is a hydroxyl group, and n is an integer of 0 to 5.

21. The allergen-inhibiting fibers according to claim 20, wherein only one of the substituent groups R is a hydroxyl group, and all the other substituent groups R are hydrogen atoms.

22. The allergen-inhibiting fibers according to claim 19, wherein the aromatic hydroxy compound is obtained by polymerizing or copolymerizing a monomer having at least one of substituent groups represented by the general formulas (1) to (6): 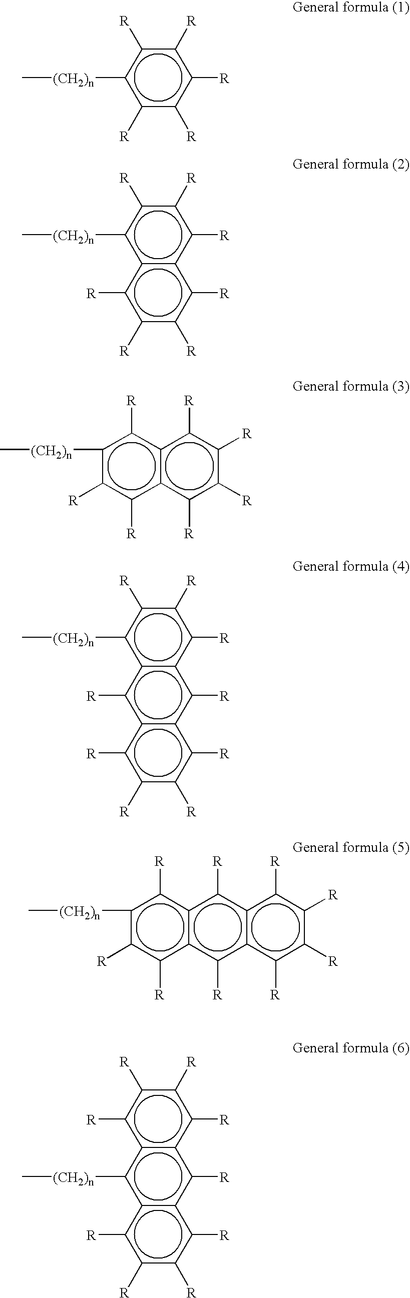
wherein R is a hydrogen atom or a hydroxyl group, and at least one R is a hydroxyl group, and n is an integer of 0 to 5.

23. The allergen-inhibiting fibers according to claim 22, wherein only one of the substituent groups R is a hydroxyl group, and all the other substituent groups R are hydrogen atoms.

24. The allergen-inhibiting fibers according to claim 19, wherein the aromatic hydroxy compound is an aromatic heterocyclic hydroxy compound.

25. Allergen-inhibiting fibers which exhibit an allergen-inhibiting effect in an atmosphere of an absolute humidity of not higher than 50 g/m³.

26. The allergen-inhibiting fibers according to claim 25, which comprises an allergen inhibitor and fibers capable of forming a reaction field capable of causing interaction with allergens by gathering water molecules in the air.

27. The allergen-inhibiting fibers according to claim 26, wherein the fibers contain a hygroscopic compound.

28. The allergen-inhibiting fibers according to claim 26, wherein the fibers are hygroscopic fibers.

29. The allergen-inhibiting fibers according to claim 26, wherein the surface of the fibers has a pH value of 6 or more.

30. The allergen-inhibiting fibers according to claim 26, wherein the surface of the fibers contains an alkali metal oxide or alkaline earth metal oxide.

31. The allergen-inhibiting fibers according to claim 26, wherein the surface of the fibers contains an alkali metal hydroxide or alkaline earth metal hydroxide.

32. An allergen-inhibiting sheet comprising an allergen inhibitor comprising at least one compound selected from the group consisting of an aromatic hydroxy compound, an alkali metal carbonate, alum, lauryl benzene sulfonate, lauryl sulfate, polyoxyethylene lauryl ether sulfate, and a divalent or more sulfate having either or both of a polyoxyethylene chain and a polyethylene chain in the molecule thereof contained in a base sheet.

33. An allergen-inhibiting sheet comprising an allergen inhibitor comprising a phosphate and either or both of zinc sulfate and lead acetate contained in a base sheet.

34. An allergen-inhibiting sheet comprising an allergen inhibitor comprising an aqueous solution containing aluminum sulfate and at least one sulfate selected from the group consisting of sodium sulfate, potassium sulfate, ammonium sulfate and thallium sulfate contained in a base sheet.
